(19) United States
(12) Patent Application Publication
(10) Pub. No.: US 2009/0240161 A1
(43) Pub. Date: Sep. 24, 2009

(54) SYSTEM INCLUDING METHOD AND DEVICE FOR IDENTIFICATION AND MONITORING OF PULMONARY DATA

(75) Inventors: George Sutton, La Jolla, CA (US); Mark Whitebook, Capistrano Beach, CA (US)

Correspondence Address: DLA PIPER LLP (US), 4365 EXECUTIVE DRIVE, SUITE 1100, SAN DIEGO, CA, 92121-2133, US

(73) Assignee: PULMONARY DATA SYSTEMS, INC., San Diego, CA (US)

(21) Appl. No.: 12/398,939

(22) Filed: Mar. 5, 2009

Related U.S. Application Data
(60) Provisional application No. 61/034,099, filed on Mar. 5, 2008.
(60) Provisional application No. 61/090,541, filed on Aug. 20, 2008.

(51) Int. Cl. A61B 5/087

(52) U.S. Cl. 600/538

(57) ABSTRACT

The invention relates to a method and device including a system for identification and monitoring of pulmonary data. The invention allows for the collection of pulmonary function test data as well as the ability to compare and correlate newly collected data with historic patient data. The invention also allows for the ability to identify individual patients based on the analysis of pulmonary characteristics unique to the individual, such as measures of lung function to ensure integrity of a patient's historical data.

### CROSS REFERENCE TO RELATED APPLICATION(S)

This application claims the benefit of priority under 35 U.S.C. §119(e) of U.S. Ser. No. 61/034,099, filed Mar. 5, 2008; and the benefit of priority under 35 U.S.C. §119(e) of U.S. Application Ser. No. 61/090,541, filed Aug. 20, 2008. The disclosure of each of the prior applications is considered part of and is incorporated by reference in the disclosure of this application.

### BACKGROUND OF THE INVENTION

1. Field of the Invention

This invention relates generally to a system including methods and devices for monitoring, storing and reporting medical information of an individual. More specifically, the invention provides a system for pulmonary function test data monitoring and analysis. Statistical methods are described for use in components of the system to ensure data integrity through identification and monitoring of pulmonary function test data.

2. Background Information

Asthma is a chronic condition involving the respiratory system. During an asthmatic episode, the airway constricts, becomes inflamed, and is lined with excessive amounts of mucus, often in response to allergens or other triggers. Asthmatic episodes are characterized by airway narrowing causing symptoms such as wheezing, shortness of breath, chest tightness, and coughing. While most asthma attacks are not life threatening, some attacks may be severe and life threatening, even leading to death.

According to the American Lung Association, approximately 22 million Americans suffer in varying degrees from different forms of asthma. Approximately 3.8 million American children had an asthma attack in the past year. Asthma accounts for an estimated 14.5 million lost work days a year for people over 18 years of age and 14 million lost school days for children ages 5-17. In 2007 alone, nearly 11.5 billion dollars were spent in total in the United States on asthma-related costs. Despite advances in the treatment of asthma, the morbidity and mortality of the disease has increased significantly during the past several years. Moreover, asthma continues to present significant management problems for patients trying to cope with the disease on a day-to-day basis and for physicians providing medical care and treatment.

The symptoms of asthma can usually be controlled with a combination of drugs and environmental changes, but require constant monitoring, for example, by administering pulmonary function tests. Pulmonary function tests may be performed for a variety of reasons, such as to diagnose certain types of lung disease (especially asthma, bronchitis, and emphysema), find the cause of shortness of breath, and measure whether exposure to contaminants at work affects lung function. Pulmonary function tests are routinely performed to assess the effect of medication or measure progress in disease treatment. Efficient asthma management requires daily monitoring of respiratory function. Pulmonary function tests, also known as spirometry tests, are a group of tests that measure how well the lungs take in and release air. In a spirometry test, a patient breathes into a mouthpiece that is connected to an airflow measurement device, known as a spirometer. The spirometer records the amount and the rate of air that is breathed out over a period of time.

Asthma is a chronic disease with no known cure. Substantial alleviation of asthma symptoms is possible via preventive therapy, such as the use of bronchodilators and anti-inflammatory agents. Asthma management is aimed at improving the quality of life of asthma patients. Asthma management presents a serious challenge to the patient and physician, as preventive therapies require constant monitoring of lung function and corresponding adaptation of medication type and dosage. However, monitoring of lung function is not simple, and requires sophisticated systems for data monitoring.

Monitoring of lung function is viewed as a major factor in determining an appropriate treatment, as well as in patient follow-up. Preferred therapies are often based on aerosol-type medications to minimize systemic side-effects. The efficacy of aerosol-type therapy is highly dependent upon patient compliance, which is difficult to assess and maintain, further contributing to the importance of lung-function monitoring.

In-home/doctor office monitoring of asthma severity is especially useful for detecting diminished lung function before serious respiratory symptoms become evident. By identifying diminished lung function before clinical symptoms develop, a patient or physician may intervene so as to prevent worsening of a condition which may otherwise result in hospitalization or death. As such, ongoing monitoring of pulmonary function is an essential part of asthma management.

Although effective for managing and treating asthma, the reliability and accuracy of conventional in-home monitoring systems are limited. Such limitations include reliance on the patient to properly perform the tests and adequate computerized clinical decision support tools for processing and evaluating test data. An especially evident limitation is the lack of measures to ensure the integrity of test data before it is incorporated into a patient's historical profile.

Unfortunately, methods and devices have not yet been described for monitoring pulmonary function test data wherein the integrity of patient data is maintained by verifying the identity of a test patient using statistical analysis of pulmonary function test data. Thus, there is a need in the art for improved systems and methods for monitoring pulmonary function test data to assess the effect of medication or measure progress in disease treatment.

### SUMMARY OF THE INVENTION

The present invention is based, in part, on the discovery of statistical methods for analyzing data generated by a pulmonary function test useful to ensure the identity of a test patient, to prevent accidental mixing of data and maintain historical data integrity. Accordingly, the present invention provides a system including methods and devices useful for identifying and maintaining pulmonary function test data.

In one embodiment, the present invention provides methods for performing a pulmonary function test including verifying identity of a test patient to ensure integrity of historical data of a patient. The method includes comparing pulmonary function test data output for a test patient with reference data of a patient using statistical analysis, thereby verifying the identity of the test patient as the patient before the data is further processed or transmitted.

In one aspect, the statistical analysis includes: (a) identifying a peak flow value of an airflow curve generated from data output for a test patient; and (b) comparing the peak flow value to a peak flow value of an airflow curve generated from reference data for a patient, for example, the patient identified as the one taking the test.

In another aspect, the statistical analysis includes: (a) normalizing an airflow curve amplitude generated from the data of the test patient to a standard value; (b) comparing flow-rate values on a point-by-point basis with a normalized reference curve based on reference data of the identified patient to generate point-by-point difference values; (c) squaring and then summing the point-by-point difference values; and (d) taking the square root of the sum of the squared point-by-point difference values.

In yet another aspect, the statistical analysis includes: (a) normalizing an airflow curve amplitude generated from the data of the test patient to a standard value; (b) shifting the airflow curve to overlay peak flow measurement of the airflow curve with peak flow measurement of reference data for the identified patient; (c) comparing flow-rate values on a point-by-point basis with a normalized reference curve based on reference data of the identified patient to generate point-by-point difference values; (d) squaring and then summing the point-by-point difference values; and (e) taking the square root of the sum of the squared point-by-point difference values.

In yet another aspect, the statistical analysis includes: (a) decomposing an airflow curve generated from the data output of the test patient into frequency components; (b) comparing the frequency components from step (a) with frequency components generated from reference data from the identified patient to generate point-by-point difference values; (c) squaring and then summing the point-by-point difference values; and (d) taking the square root of the sum of the squared point-by-point difference values.

In another embodiment, the present invention provides a system for monitoring and collecting pulmonary function test data of a test patient. The system includes (a) an airflow detection device; (b) a data communications server; and (c) a computer-readable media including (i) a data structure including reference data for a patient; and (ii) commands for performing a statistical algorithm comparing pulmonary function test data of the test patient to the reference data for the patient, wherein the statistical algorithm identifies the test patient as the patient. In one aspect the system further includes a computer platform, such as a personal computer or laptop.

In another embodiment, the present invention provides an airflow detection device. The device includes (a) a data structure including reference data for an identified patient; and (b) commands for performing a statistical algorithm comparing pulmonary function test data of the test patient to the reference data for a patient, wherein the statistical algorithm identifies the test patient as the patient.

### BRIEF DESCRIPTION OF THE DRAWINGS

FIG. 1 shows a graphical representation of data output of a pulmonary function test. The graph depicts airflow by plotting the instantaneous flow rate (in liters per second, along the vertical axis) as a function of time (in seconds, along the horizontal axis).

FIG. 2 shows a graphical representation of data output of a pulmonary function test. The graph depicts a plot of volume (in liters, along the vertical axis) as a function of time (in seconds, along the horizontal axis).

FIG. 3 shows a graphical representation of data output of a pulmonary function test. The graph depicts a plot of the instantaneous flow rate (in liters per second, along the vertical axis) as a function of volume (in liters, along the horizontal axis).

FIG. 4 shows a graphical representation of the plot of output voltage versus the airflow (standard liters per minute) for a Honeywell model AWM720P1 air sensor.

FIG. 5 shows a schematic representation of an airflow measurement device.

FIG. 6 shows a graphical representation of data output of five pulmonary function tests performed by single patient. The graph depicts airflow by plotting the instantaneous flow rate (in liters per second, along the vertical axis) as a function of time (in seconds, along the horizontal axis).

FIG. 7 shows a graphical representation of data output of five pulmonary function tests performed by single patient. The graph depicts a plot of volume (in liters, along the vertical axis) as a function of time (in seconds, along the horizontal axis).

FIG. 8 shows a graphical representation of data output of five pulmonary function tests performed by a single patient. The graph depicts a plot of the instantaneous flow rate (in liters per second, along the vertical axis) as a function of volume (in liters, along the horizontal axis).

FIG. 9 shows a graphical representation of various analytical forms of pulmonary data.

FIG. 10 shows a graphical representation of pulmonary data using a modified Maxwell-Boltzmann function (equation p4).

FIG. 11 shows a graphical representation of aggregate air flow of 225 pulmonary measurements.

FIG. 12 shows a graphical representation of aggregate volume of 225 pulmonary measurements.

FIG. 13 shows a graphical representation of aggregate lung capacity of 225 pulmonary measurements.

FIG. 14 shows a graphical representation of coefficient trajectory tracked through a data set of 225 pulmonary measurements.

FIG. 15 shows a graphical representation of coefficient trajectory tracked through a data set of 225 pulmonary measurements.

FIG. 16 shows a graphical representation of coefficient trajectory tracked through a data set of 225 pulmonary measurements.

FIG. 17 shows a graphical representation of coefficient trajectory tracked through a data set of 225 pulmonary measurements.

FIG. 18 shows a graphical representation of coefficient trajectory tracked through a data set of 225 pulmonary measurements.

FIG. 19 shows a graphical representation of coefficient trajectory tracked through a data set of 225 pulmonary measurements.

FIG. 20 shows a graphical representation of coefficient trajectory tracked through a data set of 225 pulmonary measurements.

FIG. 21 shows a graphical representation of coefficient trajectory tracked through a data set of 225 pulmonary measurements.

FIG. 22 shows a graphical representation of coefficient trajectory tracked through a data set of 225 pulmonary measurements.

FIG. 23 shows a graphical representation of coefficient trajectory tracked through a data set of 225 pulmonary measurements.

FIG. 24 shows a graphical representation of coefficient trajectory tracked through a data set of 225 pulmonary measurements.

FIG. 25 shows a graphical representation of a typical flow rate versus volume curve, including a line segment used on the leading edge of the curve used to calculate the slope at the leading part of the curve.

FIG. 26 shows a graphical representation of a typical flow rate versus volume curve.

FIG. 27 shows a graphical representation of the first derivative of the flow rate versus volume curve of FIG. 26.

FIG. 28 shows a graphical representation of the first derivative of flow rate versus volume curves of multiple individuals.

FIG. 29 shows a histogram of correlation coefficients for the data set of FIG. 28.

FIG. 30 shows a histogram of correlation coefficients for a data set of 225 pulmonary measurements from a single individual as compared to the correlation of the derivative curve of a different user.

FIG. 31 shows a graphical representation of flow rate versus volume for the sample 1 data set.

FIG. 32 shows a graphical representation of flow rate versus volume first derivative for the sample 1 data set.

FIG. 33 shows a graphical representation of flow rate versus volume for the sample 2 data set.

FIG. 34 shows a graphical representation of flow rate versus volume first derivative for the sample 2 data set.

FIG. 35 shows a graphical representation of flow rate versus volume for the sample 3 data set.

FIG. 36 shows a graphical representation of flow rate versus volume first derivative for the sample 3 data set.

FIG. 37 shows a graphical representation of flow rate versus volume for the sample 4 data set.

FIG. 38 shows a graphical representation of flow rate versus volume first derivative for the sample 4 data set.

FIGS. 39-118 show histograms of various correlations of samples 1-4 data sets.

### DETAILED DESCRIPTION OF THE INVENTION

The present invention is based in part, on the discovery of statistical methods for analyzing data generated by a pulmonary function test useful to ensure the identity of a test patient, to prevent accidental mixing of data and maintain historical data integrity. Accordingly, the present invention provides a system including methods and devices useful for identifying and maintaining pulmonary function test data.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an” and “the” include plural references unless the context clearly dictates otherwise. Thus, for example, references to “the method” includes one or more methods, and/or steps of the type described herein which will become apparent to those persons skilled in the art upon reading this disclosure, and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention relates to a comprehensive system for monitoring and analyzing pulmonary function test data for patients with chronic lung diseases, such as asthma. The system may include an airflow measurement device, computer platform and data communications server. When incorporated, the components form a complete measurement, data archive/retrieval and analysis system.

The system described herein measures a patient's lung function and formats the resulting data using standard key metrics employed in a typical pulmonary function test. The standard pulmonary function test includes a measure of Forced Vital Capacity (FVC), Forced Expiratory Volume in One Second (FEV1), FEV1/FVC, and Peak Flow Rate (PEFR).

FVC is a measure of the patient's total expiratory lung volume with results given in units of liters.

FEV1 is a measure of the volume of air forced from the lungs in the first second of the test; results are given in units of liters.

FEV1/FVC is the ratio of the one-second volume (FEV1) divided by the total forced vital capacity (FVC); the result is a scalar fraction (no units).

PEFR is a record of the highest (peak) flow attained in the course of a single “blow” test; results are given in units of liters/second.

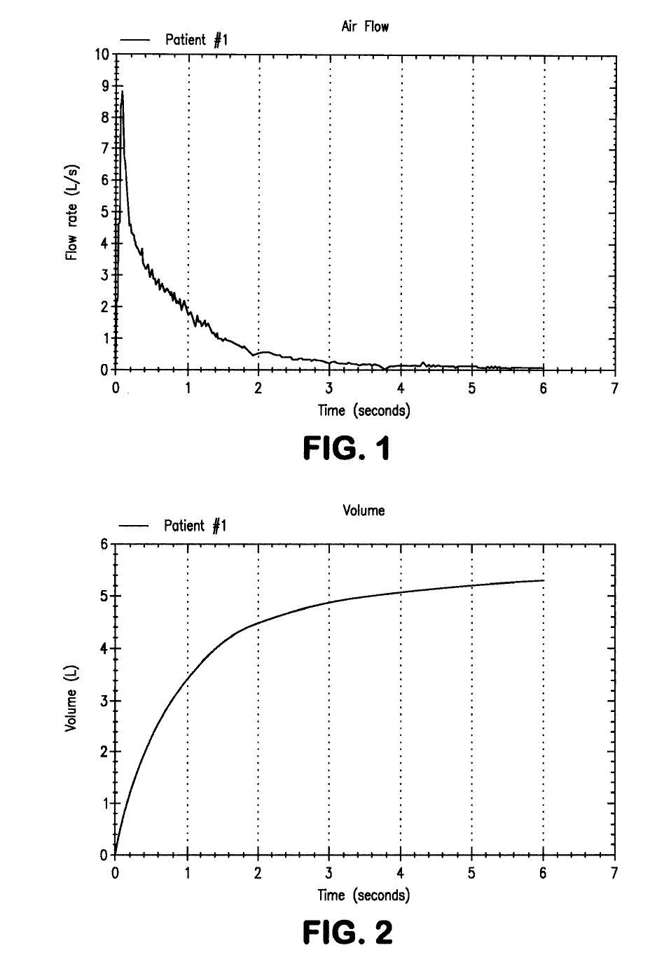

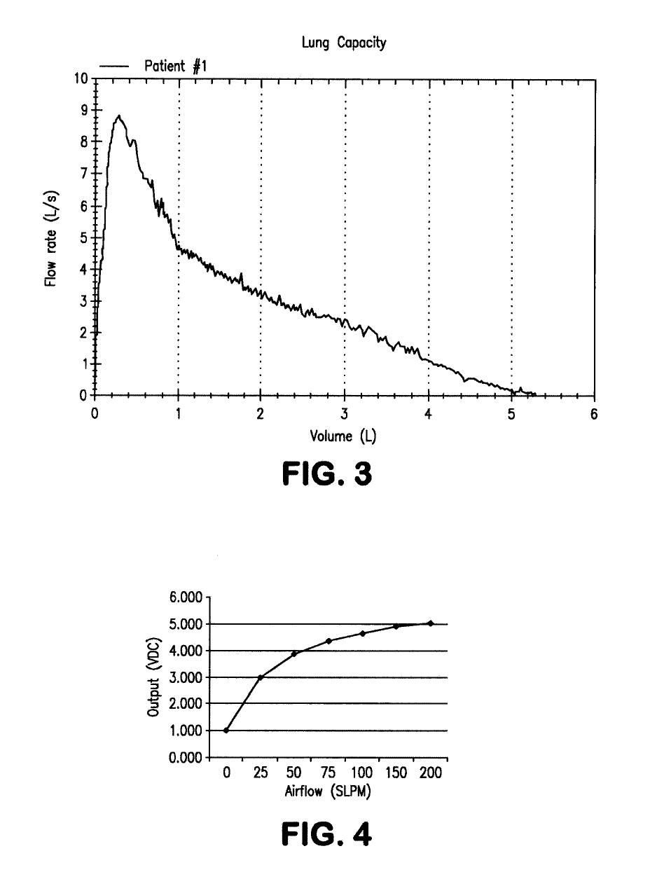

Typical graphical output of a single pulmonary function test are shown in FIGS. 1, 2 and 3 showing airflow, volume, and lung capacity graphs respectively and having FEV1: 3.42, FVC: 5.29, FEV1/FVC: 0.65 and PEFR: 8.81. FIG. 1 showing airflow, is the direct graphical representation of flow test data depicting the instantaneous flow rate (in liters per second, along the vertical axis) as a function of time (in seconds, along the horizontal axis). The airflow graph shown in FIG. 1 has a typical shape, with peak flow (in this case, 8.81 L/s) occurring in the first fraction of a second after the “blow” commences, followed by a region of rapidly declining flow, and finally tailing off to a near-zero flow rate over the last couple of seconds.

The system of the present invention may be configured to allow multiple users, each of whom log on with a unique identifier. This is due, in part, because it is not uncommon to have more than one patient in a household being monitored for pulmonary function; e.g., two or more siblings with pediatric asthma. Typically, results for each patient are tagged and stored according to an assigned user ID to keep each patient's records uncontaminated with data from another user. However, it is common for patients to make critical log-in errors for a variety of reasons, such as, due to inattention, fatigue, age and the like.

Accordingly, the present invention is based, in part, on the discovery that the identity of a patient may be verified by applying statistical algorithms to the output data of a pulmonary function test. This provides for the maintenance of data integrity and prevents accidental mixing of patient data. The statistical algorithms may be performed on the data to “flag” results that do not to match the patient's normal “baseline” data. The test results that are flagged by the algorithms result in the patient being prompted, for example, in an on-screen display message, to confirm their identity before the new test data is added to the historical database of the patient currently identified as being logged in.

As used herein, “match” refers to the similarity between particular portions of two or more data sets as determined by the statistical algorithms provided herein. Matching data sets are those in which a statistical algorithm of the present invention determines to be nearly identical and thus generated from the same individual. However, the threshold level for determining whether two or more data sets “match” may be increased or decreased.

As used herein, “data” refers to various forms of data generated or derived from the pulmonary function test. In one aspect, data refers to the output of the pulmonary function test before the output is manipulated to derive the four key output metrics (FVC, FEV1, FEV1/FVC and PEFR). In this aspect, the data may be described as a string of high-resolution digital numbers, each of which represents the patient's instantaneous expiratory flow rate (in units of standard temperature and pressure (STP) liters-per-second) as measured 1,024 times per second over a test duration of typically up to six seconds. However, the flow rate may be measured more than or less than 1,024 times per second over the duration of the test if desired. Data acquisition is automatically triggered by the flow rate rising above some very low static “floor” value, so that data is only being stored when needed.

The absolute values of the airflow curve shown in FIG. 1 may vary over calendar time for a given patient due to such factors as the effectiveness of medication or the onset of an asthma attack. For example, a patient experiencing the airflow constriction typical of an asthma attack will show a marked reduction in the peak flow figure, due to the difficulty of forcing air from the lungs. However, the present invention is based in part on the discovery that several measurable characteristics of the curve, including its general shape, are specific to an individual patient regardless of pulmonary condition.

The first such characteristic is when the peak flow occurs, relative to the onset (“trigger point”) of the test. For example, in the case of the airflow graph shown in FIG. 1, the patient's peak flow occurs within a fairly narrow window between 50 milliseconds and 70 milliseconds after the trigger, with 60 milliseconds being the nominal value. Because data may be collected at such a fast rate, for example, 1.024 kHz data rate, sub-millisecond temporal resolution is possible, allowing for differentiation between different patients on the basis of when the peak flow value occurs.

A second characteristic is the shape of the curve, in the sense of its having components that carry “signature” information that is virtually invariant for an individual patient, even at different levels of pulmonary function.

To identify signature markers for the shape of the curve, there are at least two basic approaches possible; one in the time domain, and another in the frequency domain.

The time-domain approach may be schematically described as follows.

The first step is to normalize the airflow curve amplitude to a standard value. The operation in this case would be to normalize the peak flow value to some arbitrary value, which is described as unity (“100%”). This permits comparison to other saved data from a given patient's historical data base, even if their absolute level of pulmonary function on the two dates differs. Since the peak-flow value is, by definition, the highest measurement in the data stream, all other values would be expressed as a fraction (or percentage) of the peak.

The second step is to compare the flow-rate values on a point-by-point basis to a normalized reference curve for the patient. This is a “difference” function, where the airflow value at a given point in time is subtracted from the same time-position data point in the normalized reference test. (The sign of the data, whether positive or negative, will not matter after the next step).

The third step is to square and sum the point-by-point difference values. This means that the point-by-point difference value is squared (thus making all results positive, so that “overs” and “unders” will not cancel each other out). After all the differences are squared, they are summed.

The final step is to take the square root. This step takes the square root of the sum of the square of the differences. The resulting scalar value is zero for two data streams with perfect point-by-point congruence, and takes progressively larger values for data streams with decreasing similarity.

The scalar result is then used as a measure of how closely the two data sets match one another.

A variation of the time-domain test includes both amplitude normalization and temporal offset normalization; in this case, a temporal feature other than the test's trigger-point threshold, as well as normalizing amplitudes is overlaid. Such a test can be schematically described as follows.

The first step is to normalize the airflow curve amplitude to a standard value. Again, this operation is performed as described in the first time-domain test and includes normalizing the peak flow value to some arbitrary value, which is described as unity (“100%”).

The second step is to shift the entire airflow curve to overlay the peak-flow measurement with that of the reference data. This operation would time-shift all data points equally by one-increment steps to overlay the peak-flow measurement data point of the data under test to the same point in time as the reference data. In this case, it would be important that steps involving summing and squaring, and taking the square root (steps 4 and 5 below) only be applied to data points for which there is valid data for both curves. Necessarily, some data points at both ends of the comparison data would be lost. For example, if the test data had to be shifted by 60 data points to make the peak-flow points temporally coincident, 120 data points would be sacrificed from the comparison (60 data points from the beginning, and 60 points from the end).

The third step is to compare flow-rate values on a point-by-point basis to a normalized reference curve for the patient.

The fourth step is to square and sum. As in the first time-domain test, the point-by-point difference value is squared (thus making all results positive, so that “overs” and “unders” will not cancel each other out). After all the differences are squared, they are summed.

The fifth step is to take the square root. Again as in the first time-domain test, the square root of the sum of the square of the differences is taken. The resulting scalar value would be zero for two data streams with perfect point-by-point congruence, and will take progressively larger values for data streams with decreasing similarity.

The frequency-domain analysis method does not require any pre-normalization of data, as the technique relies on performing a Fourier Analysis of the data (which typically normalized output results to a single spectral component of the data, usually the amplitude of the fundamental frequency).

Fourier Analysis is a numerical method for decomposing a complex waveform into its constituent frequency components; the lowest-frequency Fourier spectral component of a waveform is referred to as the fundamental frequency, and all other frequency components are expressed as integer multiples of that fundamental frequency. In the case of the typical pulmonary function airflow data, the significant high-harmonic frequency content extends quite far out (since a rapidly-spiking-and-reversing data segment like the peak-flow event by definition has high-frequency spectral components).

The output of Fourier Analysis is a table of amplitude values ascribed to each discrete frequency component. The values on a frequency-by-frequency basis can be compared between the data under test and the stored “reference” data for a given patient. Comparison may be done in many ways, for example, a root sum square comparison of the amplitude data may be performed.

As used herein, “reference data” is data generated for a patient that serves as the basis of the comparison. The reference data may be initially collected in controlled conditions, for example, under the guidance of a qualified clinician. An example reference data package may be an average of several “blow” samples (e.g., over 6), taken five minutes apart, to allow for recovery time. It is anticipated that several sets of reference data will be taken. For example one set representing “pre-medication” (before administering a fast-acting bronchodilation inhaler, such as ALBUTEROL™), and another “post-medication” set, taken after bronchodilation (since both types of data will typically be collected from a patient).

The system for monitoring and collecting pulmonary data described herein may include an airflow measurement device, computer platform and data communications server.

Accordingly, in one embodiment, the present invention provides a system for monitoring and collecting pulmonary function test data of a test patient. The system includes (a) an airflow detection device; (b) a data communications server; and (c) a computer readable media including (i) a data structure including reference data for a patient; and (ii) commands for performing a statistical algorithm comparing pulmonary function test data of the test patient to the reference data for a patient, wherein the statistical algorithm identifies the test patient as the patient. In one aspect the system further includes a computer platform, such as a personal computer or laptop.

In another embodiment the present invention provides an airflow detection device. The device includes (a) a data structure comprising reference data for an identified patient; and (b) commands for performing a statistical algorithm comparing pulmonary function test data of the test patient to the reference data for a patient, wherein the statistical algorithm identifies the test patient as the patient.

As used herein, the term “data structure” is intended to mean a physical or logical relationship among data elements, designed to support specific data manipulation functions. The term can include, for example, a list of data elements that can be added, combined, compared or otherwise manipulated, such as pulmonary function test data. The data structure may include the reference data or historical data for a patient, such that multiple data sets for an individual, or multiple data sets for multiple individuals may be statistically manipulated.

As used herein, the term “substructure” is intended to mean a portion of the information in a data structure that is separated from other information in the data structure such that the portion of information can be separately manipulated or analyzed. The term can include portions subdivided according to function of time for example. The term can include portions subdivided according to computational or mathematical principles that allow for a particular type of analysis or manipulation of the data structure.

Software to implement a method of the invention can be written in any well-known computer language, such as Java, C, C++, Visual Basic, FORTRAN or COBOL and compiled using any well-known compatible compiler. The software of the invention normally runs from instructions stored in a memory on a host computer system or electronic device. A memory or computer readable medium can be a hard disk, floppy disc, compact disc, magneto-optical disc, Random Access Memory, Read Only Memory or Flash Memory. The memory or computer readable medium used in the invention can be contained within a single computer or distributed in a network. A network can be any of a number of conventional network systems known in the art such as a local area network (LAN) or a wide area network (WAN). Client-server environments, database servers and networks that can be used in the invention are well known in the art. For example, the database server can run on an operating system such as UNIX, running a relational database management system, a World Wide Web application and a World Wide Web server. Other types of memories and computer readable media are also contemplated to function within the scope of the invention.

A database or data structure of the invention can be represented in a markup language format including, for example, Standard Generalized Markup Language (SGML), Hypertext markup language (HTML) or Extensible Markup language (XML). Markup languages can be used to tag the information stored in a database or data structure of the invention, thereby providing convenient annotation and transfer of data between databases and data structures. In particular, an XML format can be useful for structuring the data representation of reactions, reactants and their annotations; for exchanging database contents, for example, over a network or internet; for updating individual elements using the document object model; or for providing differential access to multiple users for different information content of a data base or data structure of the invention. XML programming methods and editors for writing XML code are known in the art.

The airflow measurement device is used to collect pulmonary function test data from the patient. It is suitable for use by the patient in the home or in the doctor's office. In one embodiment, the airflow measurement device includes a sensor subsystem and an embedded microprocessor.

While the methods and devices of the present invention are suitable for monitoring and analyzing pulmonary function test data, the invention described is also suitable for other applications. For example, in another embodiment, the methods and devices described herein may be incorporated into breathalyzers, such as, car breathalyzers known as Breath Alcohol Ignition Interlock Devices (BAIIDs). Current ignition interlock devices are capable of determining a person's breath alcohol content (BrAC), but lack the ability to distinguish whether the correct or intended person is blowing into the device. Accordingly, a device of the present invention would not only be capable of determining a person's breath alcohol content, but also ensure the identity of the person blowing into the device. This would allow a car with an ignition interlock device to require that the person for whom the interlock device was issued be present and have a BrAC below a preset level.

The embedded microprocessor(s) subsystem of the airflow measurement device imparts functionality to the device. In one aspect, it contains the sensor subsystem, data converter, a microprocessor, a real time clock, and a very simple on-board user interface. In another aspect, the device includes the computer readable media including commands for performing the statistical algorithms of the present invention and/or data structure including reference data. The sensor system monitors the pulmonary function test output of the patient (a ‘blow’). The data converter creates a digital representation of the sensor output, and packages it with time-of-day and patient information to create a ‘data set’ per blow, (which is the basis of the monitoring system). The microprocessor may manage the clock, data collection and user interface.

In another aspect, the airflow measurement device may include, an airflow sensor, interface board, microprocessor, display, user input device, power supply, and housing. Several commercially available airflow sensors are available and may be utilized in the measurement device, such as the model AWM720P1 air sensor manufactured by Honeywell. Additionally, suitable microprocessors are also commercially available, such as the model C8051F124 microprocessor development board manufactured by Silicon Laboratories.

An interface board suitable for incorporation into the airflow measurement device is generally a printed circuit board capable of performing specific functions. The principal functions include: (1) providing signal scaling and buffering of the sensor signal to the microprocessor's analog-to-digital converter (ADC); (2) providing a stable DC reference voltage for the ADC; (3) providing a real-time-clock (RTC) source to keep track of date, day, and time (battery-backed, so that the data remains accurate even when the system is shut down); (4) providing regulated DC power for the sensor; (5) providing regulated DC power for the microprocessor; (6) providing regulated DC power for the RTC; (7) buffering the signals from microprocessor to display; (8) buffering the signals from keypad to microprocessor; and (9) providing audio feedback and cues.

The display utilized in the airflow measurement device may be of virtually any type suitable for use with an electronic device. For example, the display may be built into the device or linked to the device via a hardline connection or remote wireless connection. In one aspect the display is a built in LCD having resolution of 320×240 pixels. However, the display may be configured for high resolution, such as XVGA technology.

As used herein, user input device refers to any device suitable for linkage (hardline or wireless) to an electronic device to provide a means of input. For example, such devices include keyboards and mice. In one aspect, the user input device is a keyboard incorporating a 10-digit number pad.

The power supply for use with the user input device may be any commercially available supply capable of converting AC to DC. In one aspect the supply is a self-contained wall-plug mounted AC to DC switching supply, rated at 12 Vdc, 500 mA output.

The airflow measurement device may be configured for different applications and venues in a number of ways. For example, the device may be configured for direct or remote connection to a computer platform (e.g., a personal computer). In this configuration, the data generated is transmitted directly to the computer via a telecommunications device.

As used herein, “telecommunications device” refers to any device suitable for transmission of computer-generated data. For example, such devices may include any hardline cable used for direct linkage to a computer or electronic device for transmission of data (e.g., serial, parallel, universal serial bus, and the like). Accordingly, in one aspect, the airflow measurement device is directly connected to a computer via a serial communications output for communicating with the computer platform. There may be redundant parametric data presentation on the device and on the personal computer connected to the device. In addition to the parametric data, the computer platform may also display a graphical representation of the measured data. The real time clock is used to keep track of the date and time of different ‘blows’.

In another aspect, the device may also be configured as a standalone device with data memory for storage of data. Additionally, the data memory may be removable for convenient transport where it may be accessed by a suitable device for retrieving stored data. Accordingly, any standard type of data memory is envisioned for use with the device, such as CD-ROM, hard drive, floppy disk, memory card, SDI card, flash drive and the like. As such, the airflow measurement device with removable memory may be suitable for patients with no personal computer or internet access. For example, the device may be used to collect patient data on a periodic basis (daily), and store the data on removable media for the doctor or some other facility to upload to another component of the system, such as a data communication server, described herein, on a weekly/monthly basis.

As used herein, telecommunications device also refers to devices suitable for remote access or connection, such as wireless devices. Accordingly, in another aspect, the airflow measurement device may be configured for remote connection to a computer or network. In one aspect the airflow measurement device is configured with built in networking capability, which may be suitable, for example, for patients with either telephone or internet connectivity in the home, but with no access to a personal computer. Accordingly, the device may connect directly to another component of the system, such as the digital communications server during or after each patient blow. As such, two-way communication with the pulmonary data system is established so that alerts could be sent to the device from the system during daily data collection sessions. All communications via the internet are encrypted through a secure socket layer and utilize an encryption key seed based on the unique device serial number and other data in the data collection device.

The pulmonary data system described herein, may also include a computer platform, for example, a personal computer or laptop. The functions of the computer in the system are mainly focused on data acquisition and manipulation and display. As such the functions may include, use as a telecommunications device, interpretation and storage of data, graphical interaction with users for collecting data, such as children (e.g., games for kids).

The personal computer of the pulmonary data system may provide communication to either a removable storage device (such as a memory stick) or directly to the data communications server via a telephone line utilizing a modem or via the Internet using a broadband (Ethernet) connection (DSL, Cable Modem, WiFi modem, Satellite uplink). In the case of the storage media, data will be delivered to monitoring healthcare professionals or the attending physician on a weekly/monthly basis. The healthcare professional or the physician may use the personal computer to upload a patient's data to the data communications server.

The data interpretation is performed after data is initially screened using the algorithms provided herein. The data interpretation takes the data collected during each blow and interprets the data for all facets of a pulmonary test function output including, but not limited to, the Peak Expiratory Flow Rate (PEFR), Forced Expiratory Volume in One Second (FEV1), Forced Vital Capacity (FVC) and Ratio of volumes expelled from lungs (FEV1/FVC). Predicted values based on patient vital statistics and ratios of collected data values to those predicted values are also displayed. The medical professional may select which algorithms (those published in medical literature or the like) are used from drop down menus at system configuration time. The algorithms may be updated from published medical literature.

The personal computer may also be used for applications targeting children facilitating interest in performing tests. For example, a “Games for Kids” application that is part of the system may be targeted towards different ages of patients to make the monitoring of the pulmonary function a fun and sustainable action. This may allow the system to track compliance, and increase that compliance over the mundane task of blowing into the airflow measurement device. Compliance to medical treatment or monitoring is a major function of the pulmonary data system. With day-to-day monitoring the system's algorithms can be programmed to predict the onset of a pediatric asthma event, and warn the patient, the parent, and the physician to either change, or begin treatment prior to the patient needing to be hospitalized, or visit the emergency room.

The data communications server (DCS) of the pulmonary data system may be configured to undertake several functions. The DCS may function to (1) communicate with distributed devices; (2) interpret data sets received; (3) enable Web presentation of the data sets of select patient sets; (4) communicate notifications to distributed airflow measurement device(s); (5) facilitate compliance metrics; and (6) analyze data.

The DCS communications with devices and PCs in the field (both in-home and doctor's office) may be handled by the communication server. All communications via the internet will be encrypted through a secure socket layer, and will also utilize an encryption key seed based on the unique unit serial number and other data at the data collection device. To ensure patient confidentiality, any Web server applications may be located on a separate server.

Data sent from the measurement devices can be in various forms, such as raw output, linearized, or data derived from such sources. For example, in one aspect the data sent is discrete flow rate data points with informational headers to create unique data sets on the database server for each ‘blow’. In various aspects of the invention, data may be screened at any step using the algorithms of the present invention, for example, on the air flow measurement device, the personal computer or the DCS. Further, the algorithms of the present invention may be performed on various forms of output data, regardless of whether the data is raw, linearized, or data derived from such. Data interpretation done either at the PC or on the measurement device need not be transferred to the DCS. After data is determined to be of the correct individual, the DCS uses the data collected during each blow and interprets the data for all facets of a pulmonary test function output including, but not limited to, the Peak Expiratory Flow Rate (PEFR), Forced Expiratory Volume in One Second (FEV1), Forced Vital Capacity (FVC) and Ratio of volumes expelled from lungs (FEV1/FVC).

A key feature of the system is the ability to present patient data using a Web browser. This data can be made available to anyone with approved access. The data can be presented to the patient, patient's doctor, medical practice (multiple doctors), and medical professionals (impersonalized).

In one aspect, the patient or guardian may view their own data. This can be viewed on a day-by-day basis with interpretation results, or in a scatter graph mode that can include any number of days of data, without interpretation. In another aspect, each doctor with patients using the system may be able to access their patient's data via the web application. When a doctor logs into the system, a list of his/her patients may be displayed. The doctor can select a patient and display data in either single or multiple day modes. Each medical facility (for example, a four-doctor practice) will also be able to access all patients being treated by that particular practice in the same way a single doctor can access his/her patients. In yet another aspect, medical professionals may access data. A key feature of the system pertains to the way in which the databases are segregated. The patient name associated with the data is protected by compliance with all patient privacy regulations including the Health Insurance Portability and Accountability Act (HIPAA). The individual ‘blow’ data for all patients may be made available to medical professionals without name association. This allows a variety of different query sets into a massive database of pediatric asthma patients. The data retained may be referenced by any set of classifications, such as date of birth, height, weight, race, and sex of the patient. Additionally, data may be referenced by other information such as location. The data may be accessed and used for tracking of national and international trends. For example, a query may be to graph all data for the month of August of patients using a particular long term medication versus those who are not.

The DCS enables the system to notify users of anomalies in patient data on an ongoing basis. The system may be configured to track each patient's pulmonary function over time and can be programmed to notify the user if certain parametric are met. For example, if a patient's pulmonary function declines for a number of days at or above a certain rate (this science will be collected from medical advisors and the Asthma guidance documents published by the medical community), the system can begin notifying the appropriate medical personnel and caregivers. This notification may be done, for example, by email, fax, recorded phone message, paging device, visual and audio indicators on a particular device or component of the system, and the like. The notification may be sent to parents, doctors' offices, and the like, whoever is set up in the system to be responsible. In one aspect, the visual and audio indicators may be on the airflow measurement device and may be set to, for example, turn on a red indicator when the patient starts a collection session.

The system of the present invention may be used by doctors, drug manufacturers, and the like, to monitor compliance of each patient using the system (as opposed to assuming the patient is monitoring their pulmonary function). The system may use the same notification as when there is a parametric anomaly to remind the patient, or their guardian to help achieve compliance.

The drug manufacturer's use of compliance metrics is more to help with the data collection while monitoring the function of a treatment regimen, or drug. If the patient is supposed to ‘blow’ twice each morning, once before and once after a new medication—the system may be configured to record not only the effects of the before and after each day, but may allow for tracking of whether the regimen is being followed. This type of tracking of compliance enables the drug manufacturer to have the data on whether the drug is acting differently because of some individual effect, or because the regimen is not being followed.

The system's DCS allows multiple medical professionals to monitor and analyze data collected for each patient or groups of patients in various ways pursuant to algorithms or statistical methods as described, for example, in medical literature. Parameters for analysis may include sex, age, height, weight, race, demographic, geographic, environment and medication type.

In addition to test data, the system may further be configured to incorporate databases of records including any number of patient characteristics and details, such as a patient's physical characteristics, medical history, current health status at the start of each test, and data collected from pulmonary function tests. Such entries enable viewing of statistical analysis of patient data of a particular demographic and/or geographic set. Interested individuals may include, for example, patients, medical practitioners, health care providers, prescription drug manufacturers, and researchers. Specific queries may be performed of the analyzed data. A health care provider, for example, may want to access pulmonary function test data of a specific population segment (African-American children between the ages of 7 and 12 years) in specific geographical areas (within 5 and 10 miles of a specific location).

The system may also be configured such that a user or interested individual may perform user-defined statistical analysis. Data from pulmonary function tests may be interpreted by the DCS and input to the patient record entries of the database as values of lung volume, such as FVC, FEV1, FEV1/FVC, and PEFR.

A patient may have access to his/her personal records in a secure online environment. This allows for close monitoring of pulmonary function and of alarm criteria set by the medical practitioner. The patient can interpret real time variations in his pulmonary condition and in the case of a reduction of pulmonary function test values relative to reference values; the patient will be able to determine a course of action in time to prevent an exacerbation of symptoms.

Spirometry measurements form the basis for setting alarm criteria for patients. Once a classification of asthma severity is determined and treatment is established, then the emphasis is on assessing asthma control to determine if the goals of therapy have been met. Based on the percentage of pulmonary function test values in relation to predicted values determined by factors, such as, age, height, gender, and race an alarm criteria can be established.

However, relying only on purely numerical results for clinical decision making is a common mistake. Interpretation of data should also take into consideration other factors, such as, socioeconomic and environmental characteristics of a patient. The detailed medical history input to the database allows for additional information in determining alarm criteria. For example, a medical practitioner with school aged patients from a particular region of a city may want to tighten alarm criteria due to the high rate of morbidity and mortality due to asthma. The database can take additional factors into account allowing medical practitioners to create a more personalized set of alarm criteria in order to detect early changes in asthma disease states.

The following examples are intended to illustrate but not limit the invention.

### Example 1

### Construction and Use of the Airflow Measurement Device to Generate Clinically Significant Values of Pulmonary Function

An airflow measurement device was constructed including, an airflow sensor, interface board, microprocessor, display, user input device, power supply, and housing.

The device utilized an AWM720P1 airflow sensor manufactured by Honeywell. The AWM720P1 is Honeywell's highest-range flow sensor; it has a measurement range extending up to 200 standard liters per minute (SLPM; divide by 60 to obtain the more commonly-used measurement units of liters per second, for 3.3 LPS maximum measurable flow rate). Since the peak expiratory flow rate of a healthy grown man can be upwards of 12 LPS, it is clear that the entire airflow cannot be routed through the Honeywell sensor without driving its output signal into saturation. Thus, the technology-demonstration units employ a “flow-splitter” to apportion the total mass flow between the sensor and a “bypass,” with the majority of the flow being directed to the bypass. So long as the mass flow through the sensor is consistently representative of the total mass flow, a simple scaling factor can be implemented in the data processing to accurately equate the measured flow to the total flow.

The AWM720P1 sensor is configured as a temperature-compensated and amplified “bridge” topology. A nominal 10.0 Vdc bias applied to the sensor results in an output voltage of 1.0V at zero airflow, and 5.0V output at 200 SLPM (3.3 LPS). As shown in FIG. 4, the output-voltage versus flow-rate transfer function is highly nonlinear, and therefore requires secondary linearization in the signal-processing steps. Also shown in FIG. 4, the change in airflow per change in output voltage is quite large near the upper end of the flow range, which equates to low resolution and large uncertainties when trying to equate a specific output voltage to a given flow rate. For this reason, the “flow splitter” was configured to use only the lower half of the sensor's nominal range, where the resolution is far more favorable and the measurement uncertainty lower.

The interface board of the airflow measurement device was a custom printed circuit board. The principal functions include: (1) providing signal scaling and buffering of the sensor signal to the microprocessor's analog-to-digital converter (ADC); (2) providing a stable DC reference voltage for the ADC; (3) providing a real-time-clock (RTC) source to keep track of date, day, and time (battery-backed, so that the data remains accurate even when the system is shut down); (4) providing regulated DC power for the sensor; (5) providing regulated DC power for the microprocessor; (6) providing regulated DC power for the RTC; (7) buffering the signals from microprocessor to display; (8) buffering the signals from keypad to microprocessor; and (9) providing audio feedback and cues.

The device also incorporated a C8051F124 microprocessor development board manufactured by Silicon Laboratories. Connections from the microprocessor development board to the interface PCB were made by prefabricated ribbon cables terminated with 10-pin, two-row connectors, which are compatible with matching headers on the two PCBs.

The power supply was a low-voltage, low-current AC-to-DC plug-mounted unit, supplying 12 Vdc to the interface board. The LCD was a backlit two-row dot-matrix type device. The keypad was set up in the familiar numeric “10 key” configuration, with additional dedicated buttons for “cancel,” “function,” “clear,” and “enter” operations.

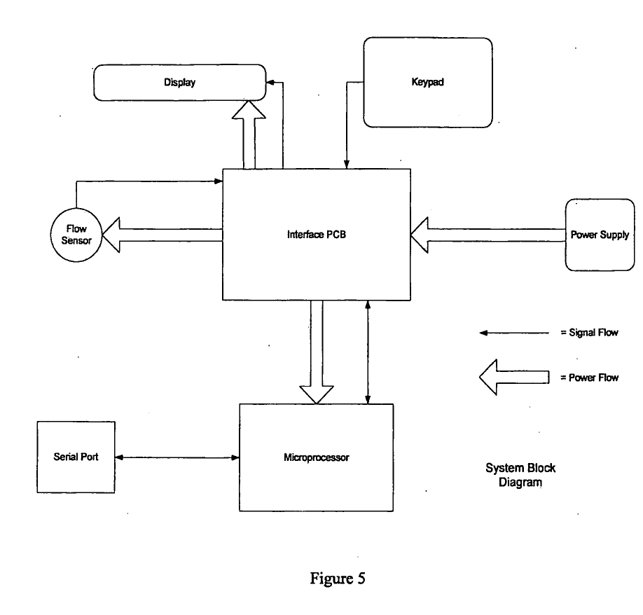

The configuration of the device is shown in FIG. 5. As shown in FIG. 5, the interface board sits at the center of the system, distributing power and coordinating signal flow. The airflow sensor receives regulated 10.0 Vdc from the interface board, and puts out a DC voltage varying between 1.0V (corresponding to zero airflow) up to 5.0V (corresponding to full-scale airflow of 3.3 LPS). The interface board divides the sensor output voltage exactly in half, buffers the signal, and delivers it to the analog-to-digital converter (ADC) input of the microprocessor. The interface board also derives a regulated and buffered reference voltage of 3.67V for the microprocessor's ADC function.

The airflow sensor's scaled-and-buffered signal voltage arrives at the microprocessor's ADC input, where it is converted from the analog domain (voltage) to a digital number, proportional to ratio of the signal voltage to the reference voltage. The ADC conversion rate is 1,024 Hz.

To make the airflow data useful, three operations are performed by the microprocessor in the digital domain (that is, after ADC conversion). First, the DC offset “baseline” must be subtracted from the measurements (the “baseline” is the measured value corresponding to the 0.5V ADC input at zero airflow). For the 12-bit ADC of the Silicon Laboratories C8051F124 processor, the 0.5V offset voltage equates to about 767 ADC counts in digital-number space.

The second operation that the microprocessor must perform on the data is to “linearize” it; that is, the inherent non-linear transfer function of the sensor must be corrected by applying the inverse function.

Using the output voltage-versus-airflow points derived for the Honeywell air sensor data-sheet table, a linearization table is created and stored in the microprocessor. Each airflow data point (6 seconds' worth of data at 1,024 Hz, or 6,144 discrete data points) in a typical patient airflow test is linearized by adding and dividing by the appropriate stored offset and slope parameters.

The third operation of the microprocessor performed on the pulmonary function test data is to apply a “coupling constant.” The coupling constant is a simple scale factor that equates the fraction of the airflow that is routed through the sensor to the patient's total airflow.

Once the data has had the DC baseline subtracted, has been linearized, and has had the coupling constant applied, it is used to develop clinically-significant displayed values.

The principal clinically-significant values calculated were the peak-flow rate (PEFR), the forced vital capacity (FVC), the one-second expiratory volume (FEV1), and the FEV1/FVC ratio.

The peak-flow rate, PEFR, is derived by searching the data for the highest flow-rate figure developed over the course of the test “blow”. This typically occurs within the first 50 to 100 milliseconds of test data.

The forced vital capacity, FVC, is the integral of the data (with respect to time) over the full six-second duration of the AirFlow test. By mathematically integrating a rate (liters per second) by time (seconds), the resulting number is the total volume of expired air, in units of liters.

The FEV1 measurement, which is the expired volume from the onset of the test through the first second, is taken by integrating the flow rate only over the time interval from zero to one second.

The ratio of FEV1 over FVC is the simple math operation of dividing FEV1 (in liters) by FVC (also in liters); the measurement units of volume drop out, leaving a dimensionless scalar.

### Example 2

### Multiple Collections of Pulmonary Function Test Data from a Single Patient Over Time

A pulmonary function test was performed by Patient #1 at five different times over the course of 2 weeks utilizing an airflow measurement device as described in Example 1.

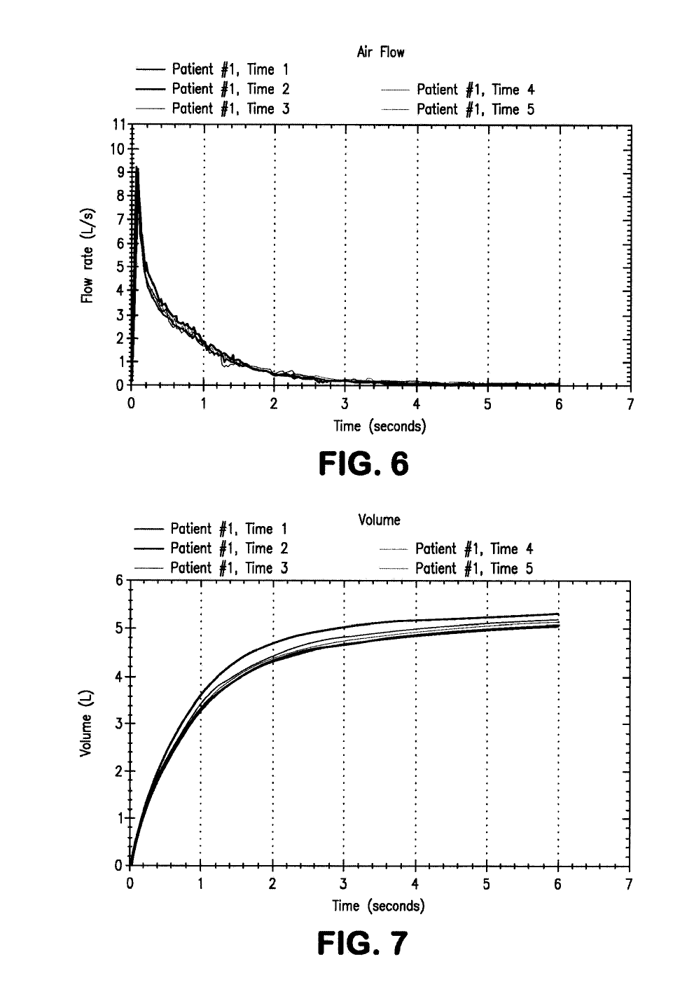

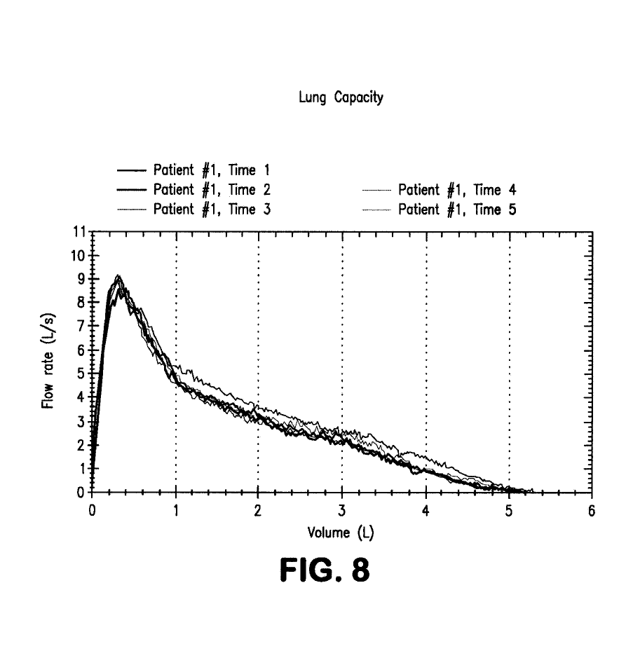

FIGS. 6, 7, and 8 show a compilation of pulmonary function test data collected for Patient #1. The figures show graphical representations of the data output showing representations of airflow, volume and lung capacity for the five repetitions of “blows” performed by Patient #1.

The graphs show signature of characteristic features and shapes that are consistent between the blows for Patient #1. The first such characteristic is when the peak flow occurs, relative to the onset (“trigger point”) of the test. For example, in the case of the airflow graph shown in FIG. 6, the patient's peak flow occurs within a fairly narrow window between 50 milliseconds and 70 milliseconds after the trigger, with 60 milliseconds being the nominal value consistently for each test. To identify additional “signature” information that is virtually invariant for an individual patient, even at different levels of pulmonary function, the data collected is further manipulated by the statistical algorithms described herein. The signature characteristics may then be compared with historical or reference data of the patient logged into the system to confirm the identity of the test patient. Data integrity is a key function for statistical analysis of the data collected for each user of the system, whether from a single device, or system wide. Compliance of the use of the monitoring device, and the ability to mark anomalous data prior to its being entered into the historical data is a key function of the system.

By application of the system's statistical and analytical ability, a patient's pulmonary function signature can be “learned” by the system, and be able to discern whether a particular data set is from the correctly identified patient, even if the test patient accidentally logs in as a different patient. The system also functions to discern bad ‘blow’ data, as opposed to compromised pulmonary function.

When the airflow measurement device is connected to a computer platform or is used as a standalone unit connected to the internet, two way communication exists with the system and the data communication server. Alerts can be sent to the patient in the event that an anomalous pattern is detected and appropriate action can be taken.

### Example 3

### Statistical Methods for Analysis of Spirometry Data

Spirometry data, was expressed as expelled air flow rate measured as a function of time (time is implicit and can be determined from the data sampling rate). This form of the data was converted into a form that expresses expelled air flow rate in liters/sec as a function of total volume, the graph of which is one common representation of human spirometry data. A parametric equation was used to represent the graph and analysis of the equation's coefficients and how these coefficients evolve over time enable the system to perform functions such as user identification, verification of data sample validity, and prediction of adverse health events.

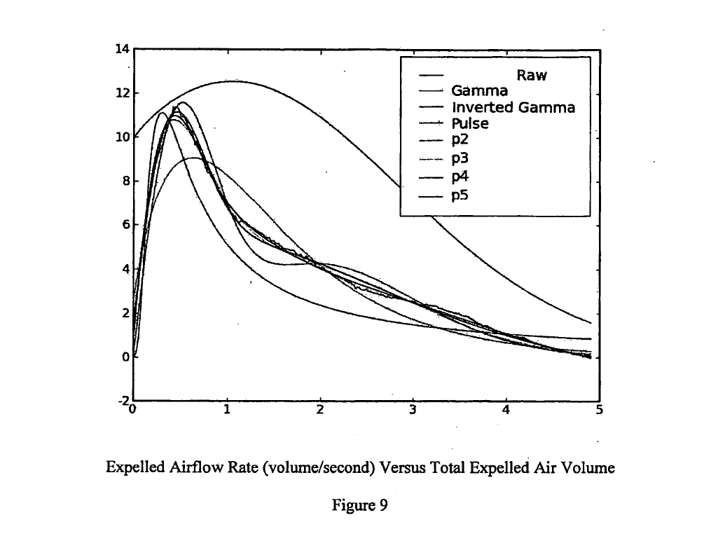

To determine an analytic form to effectively represent the measured data, efforts were directed toward matching the lung capacity graph which depicts expelled air flow rate (volume/s) as a function of total expelled air volume as shown in FIG. 9. The following types of functions were used to statistically analyze the data represented in the airflow versus volume graphs: gamma, inverted gamma, pulse, Maxwell-Boltzmann and four modified Maxwell-Boltzmann functions (p2-p5). A modified form of the function was first used to analyze the data presented in the airflow versus volume graphs. Three of the modified Maxwell-Boltzmann functions were found to be superior and provided adequate convergence robustness and quality of fit to the airflow versus volume curve. In particular modified Maxwell-Boltzmann function p4 exhibited a superior quality of fit including ideal peak matching, transition from peak to linear region, and tracking of linear region. Modified Maxwell-Boltzmann function p4 contains 8 parameters (k0-k7), values for which can be determined using a nonlinear least squares technique to provide very good matching to the measured data.

Modified Maxwell-Boltzmann function p4 is represented by the following formula:

k0*x²exp(k1*x²)+k2*x*exp(k3*x²)+k4*x*exp(k5*x)+k6*x+k7.

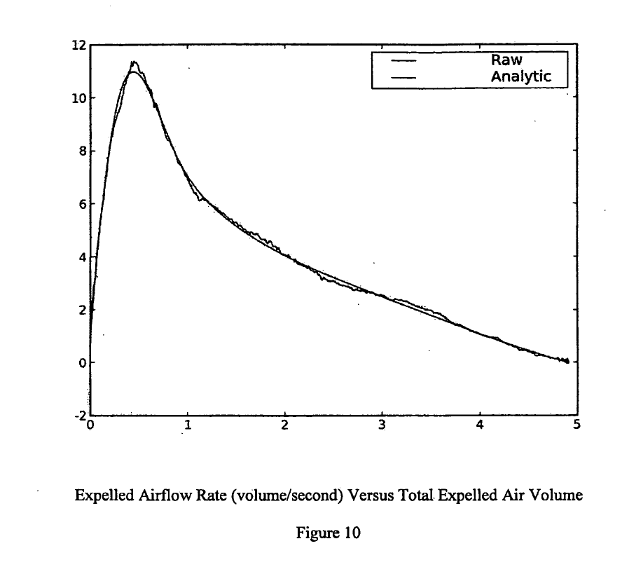

In an effort to understand the sensitivity of the representation to each of the coefficients, a fit of the data was first performed to determine the value of each of the 8 coefficients (k0,k1,k2,k3,k4,k5,k6,k7). These values give a very good fit to the data as shown in FIG. 10.

Next, each parameter was independently varied between 0.25 and 1.75 times its best fit value and the resulting family of curves was plotted. From the results, the sensitivity of the shape of each portion of the curve to variation of each the coefficients is learned.

The work described above was used to develop the ability to load and store multiple spirometry data sets from a single user and to develop the ability to analyze changes in the data sets over time. The goal was to determine if it is possible to identify trends in the data. Identified trends allows data collected in the future to predict whether or not certain events or conditions are likely to occur. The prototyping effort described above enabled the reading and analysis of a single data set.

Simultaneous analysis of multiple data sets required the development of a much more sophisticated software product prototype that enabled 1) reading in an arbitrarily sized, specifically formatted text file containing multiple spirometry data sets; 2) representing the multiple data sets with a set of dynamic data structures, classes, and methods; 3) independently determining the best fit parameters for each data set; 4) representing coefficient trajectories to enable trend identification effort; 5) developing methods to persistently store all data, including, for example, raw, derived, and fitted representations of the spirometry data sets so subsequent analysis of that data set can be performed without the penalty associated with reading in data text files and re-performing the nonlinear least squares calculation to determine coefficients for each data set.

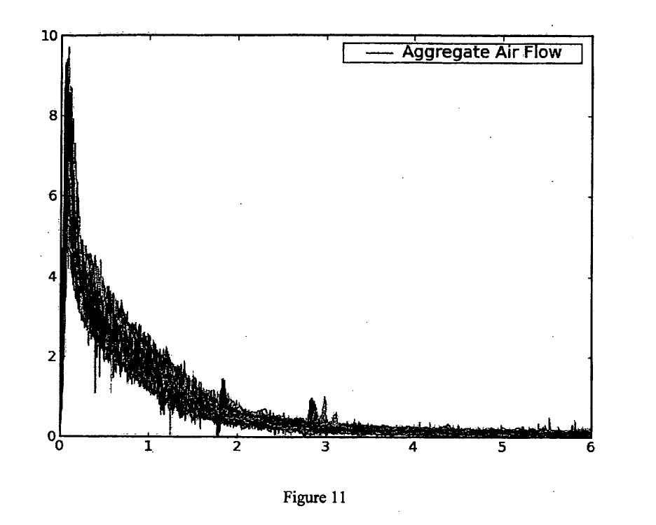

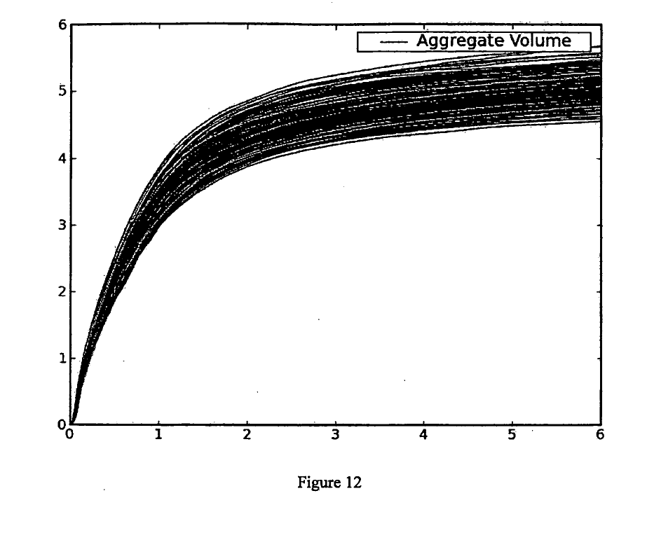

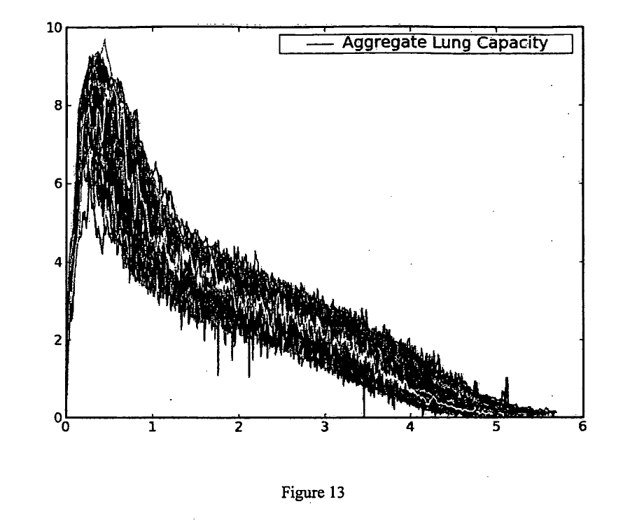

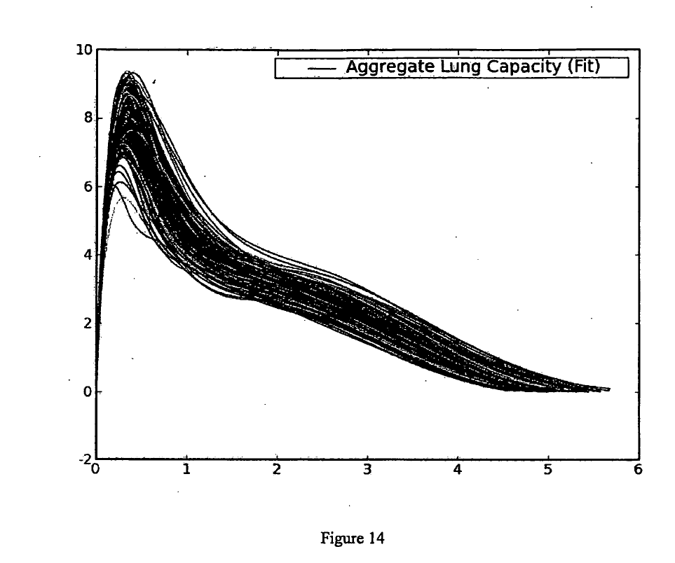

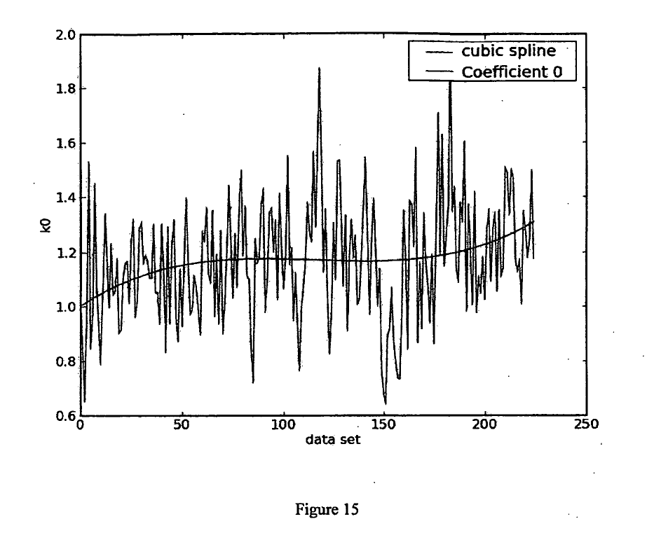

Examination of the family of curves associated with each representation of the data (FIGS. 11-14) shows the range of data values obtained over the course of 225 measurements.

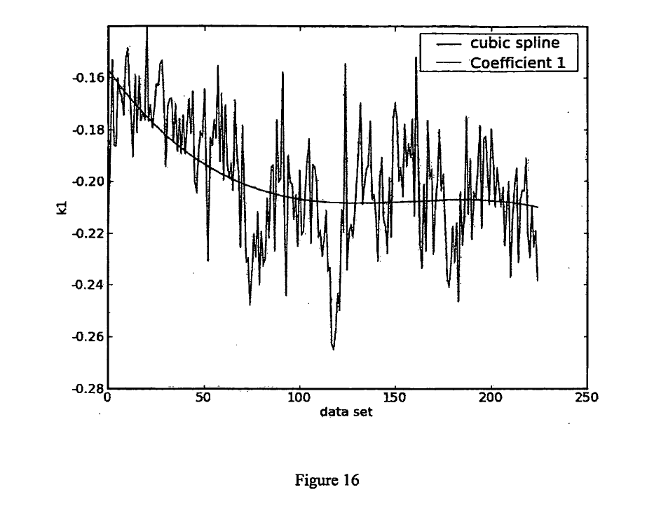

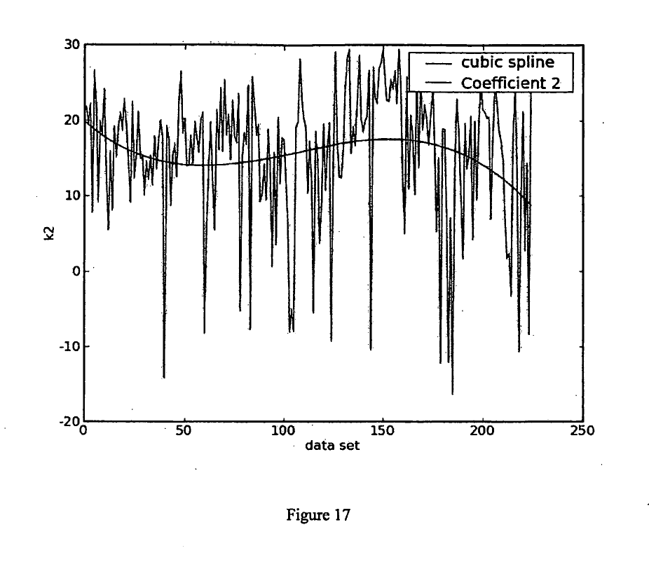

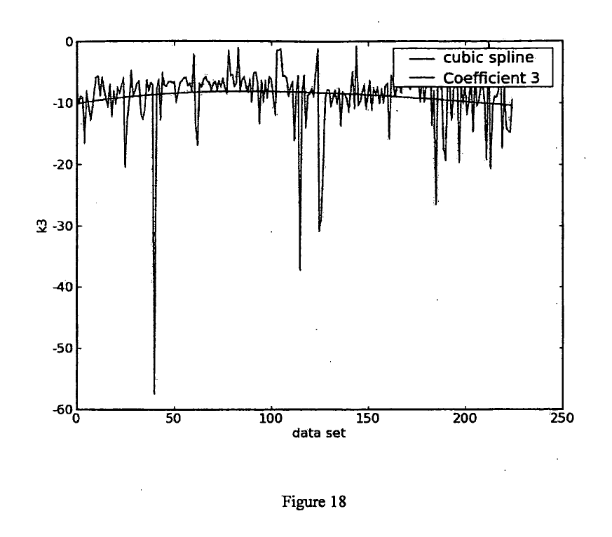

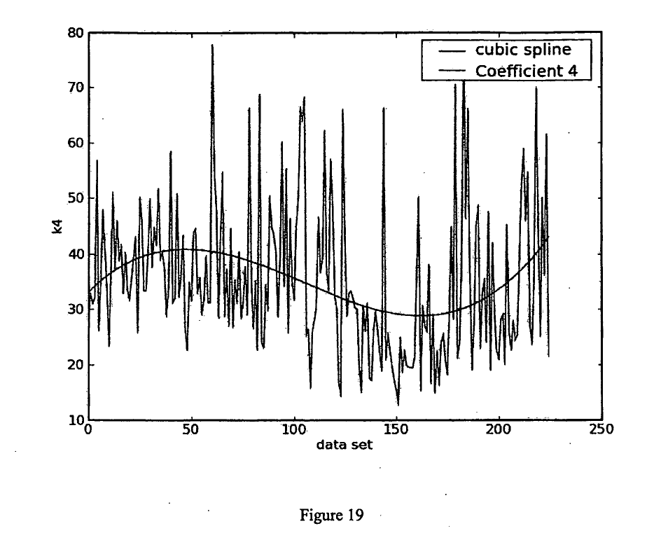

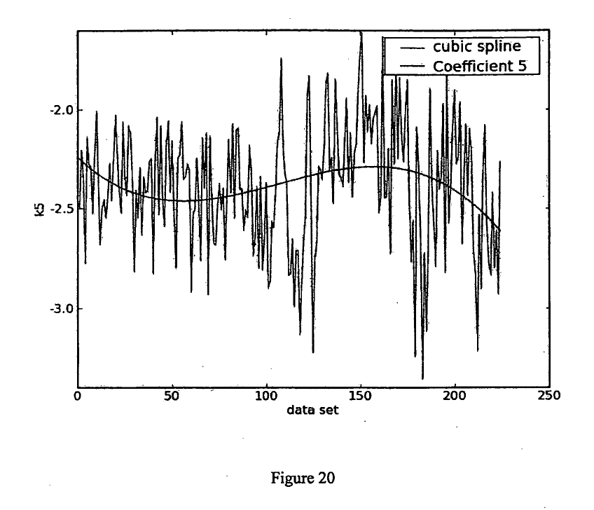

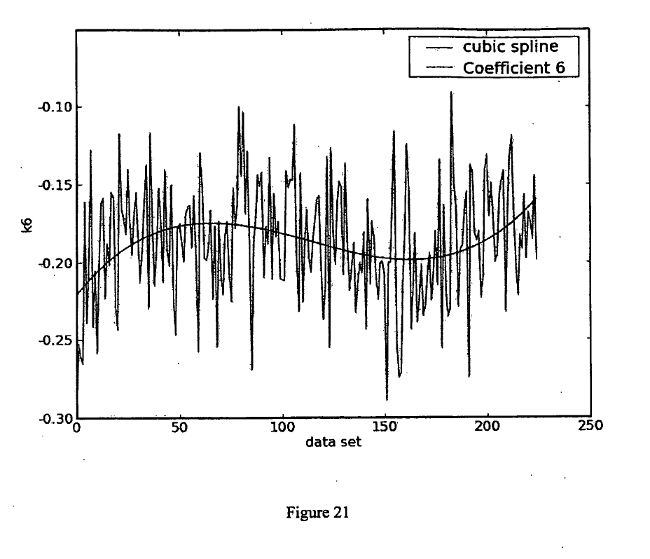

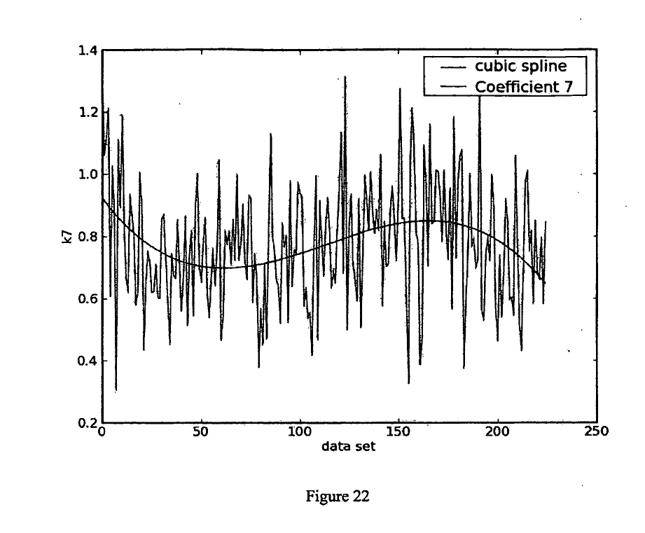

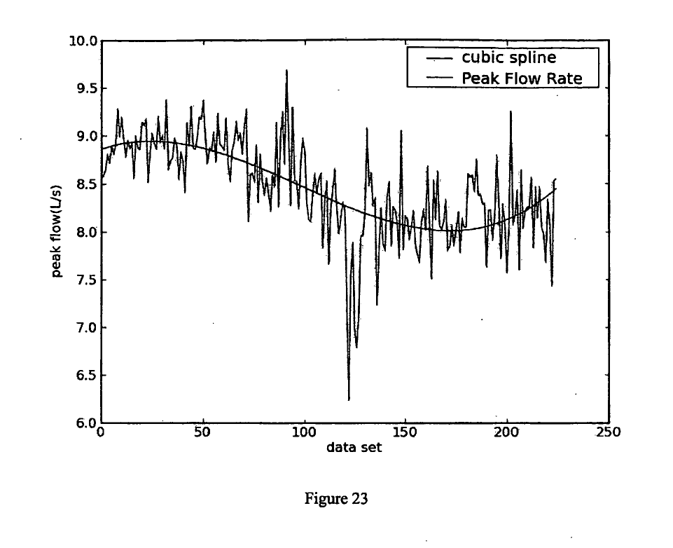

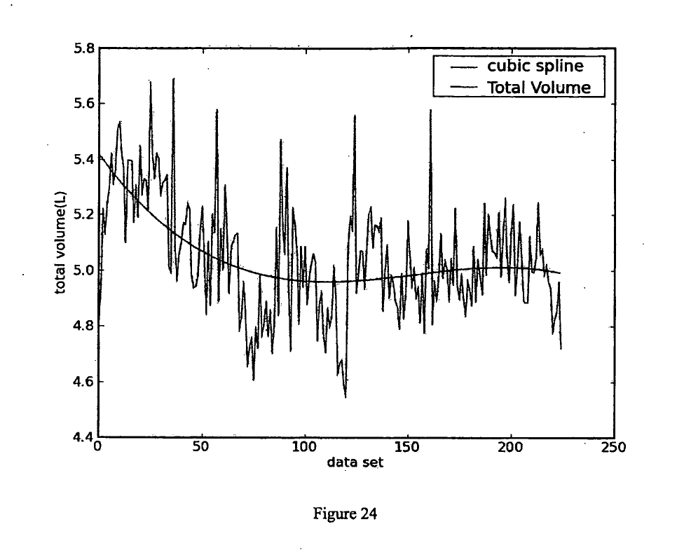

One method that is useful in identifying trends in the data across a series of measurements is to analyze the variation of the eight coefficients embodied in the equation used to fit the data along with the total expelled volume and the peak air flow rate. For this method to be effective, specific artifacts of the evolution of a coefficient or combination of coefficients need to be correlated with the occurrence of health events of interest in the human user. The coefficients are referred to as k0 through k7 in FIGS. 15-24. Peak flow rate and total volume are also shown. The smooth line that runs through the plots of coefficient data is a coarse cubic spline that is included to visually provide some notion of the general long term trajectory of the coefficient.

The next phase of analysis was directed toward analyzing certain characteristics of the flow rate versus volume curves to see if any might be used to differentiate one user's data curve from another user's, which is referred to herein as classification.

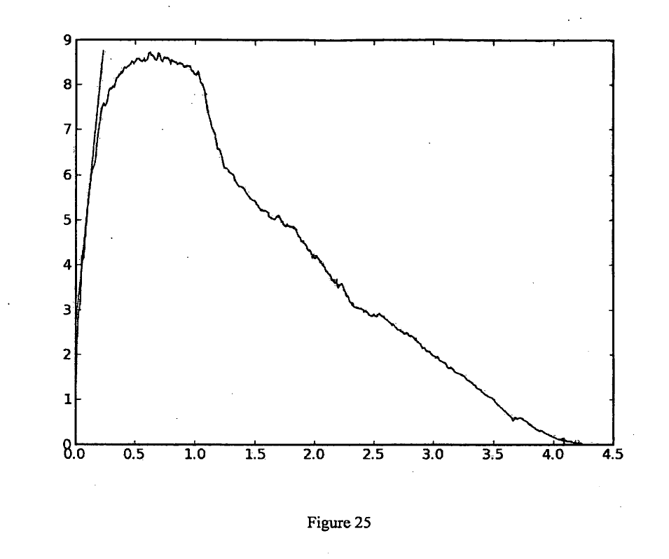

The slopes of the line tangent to the curve in the steep regions before and after the peak are a useful distinguishing characteristic. Initially the curve was split into 2 regions: from the start of data to the peak and from the peak to the end of data. Points at the ⅔ peak height of the curve on the leading and trailing regions were selected for calculation of the slope. A line segment that was used on the leading region to calculate the slope is shown in FIG. 25.

Once the ability to calculate the slopes was established, the next step was to calculate it for all curves and attempt to use it to classify curves. While implementing this step, the deficiencies of the approach became difficult to ignore, the two most egregious being 1) the arbitrary selection of the point at which the slope is calculated; and 2) the fact that 2 curves with dramatically different shapes might have identical slopes at the points chosen.

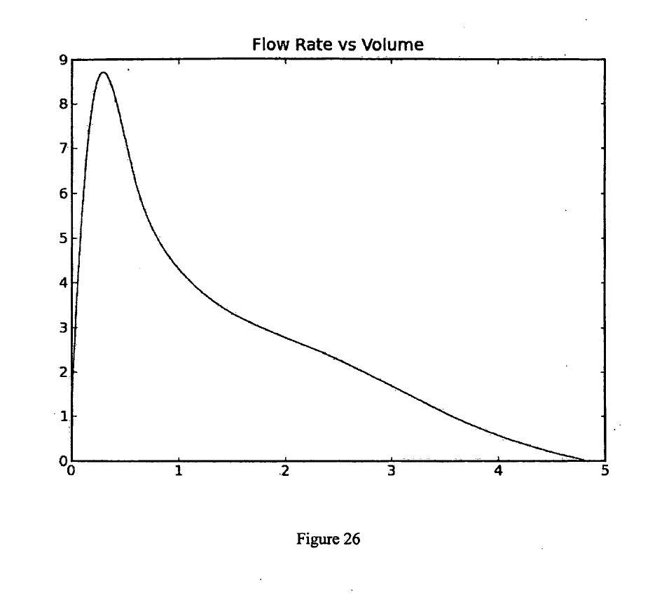

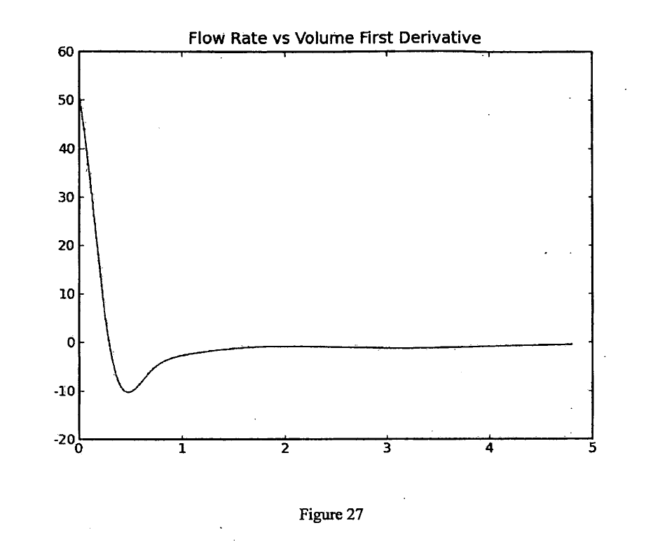

These deficiencies might be mitigated by choosing more points in the leading and trailing regions at which to calculate slopes. These slopes could then be used for classification. Extending this reasoning, the first derivative (slope) can be calculated along the entire curve and used for classification. This approach was followed. A graph of a sample flow rate versus volume curve along with its derivative curve are shown in FIGS. 26-27.

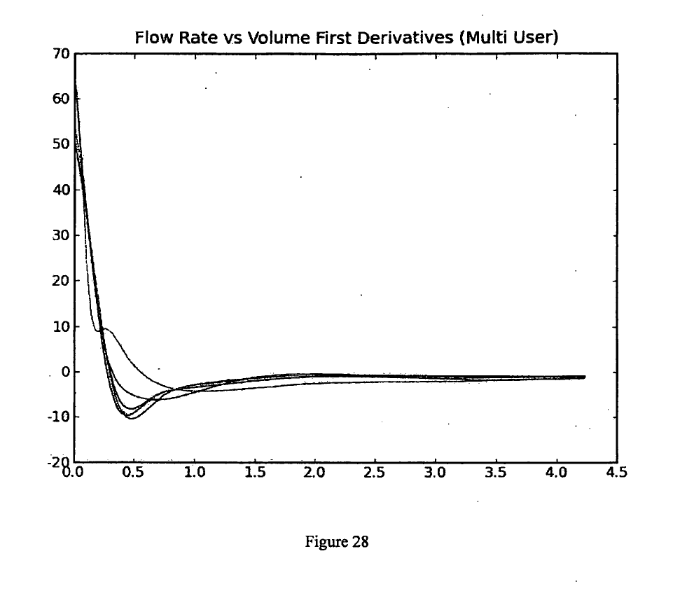

So, a set of derivative curves must be calculated for every data curve. Initially, the data was used to calculate derivatives but noise in the data appears in the derivatives as well, so the fitted data curves are used for derivative calculation. Once the capability to calculate a derivative curve for all data curves was established, a data set was created including five measurements from one user and one from a different user (multi-6). The derivative curves from this data were plotted as in FIG. 28 and examined.

Note that the shape of one of the curves above is distinguishable from the rest as an outlier having a generally different shape than the other curves. In fact, this curve corresponds to the odd user. Review of this curve suggests that it might be possible to use a statistical correlation technique to classify the derivative curves. After reviewing and testing different techniques (Pearsons product moment, Lin's concordance, Spearmans correlation, point biserial, and Kendall's tau), it was determined that Spearman's correlation coefficient provided a useful measure by which to classify the derivative curves. Spearman's correlation coefficient is defined as ρ where ρ is represented by the following formula:

$$ρ = 1 - \frac{6 \sum {d}_{i}^{2}}{n ( {n}^{2} - 1 )}$$

and di=the corresponding difference between each rank of corresponding values of x and y, and
n=the number of pairs of values subject to the constraint

$${∃}_{i , j} ( i \neq j ( {x}_{i} = {x}_{j} {y}_{i} = {y}_{j} ) ) .$$

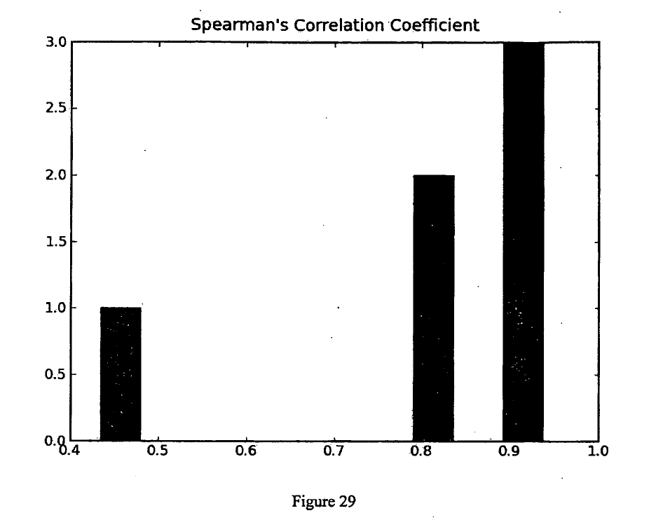

Using the multi-6 data set, Spearman's correlation coefficient was calculated to get a measure of how well each measurement correlated with the mean (in this case mean is the derivative curve determined by averaging all derivatives from a single user together). The graph of FIG. 29 shows a histogram of the correlation coefficients for the multi-6 curve.

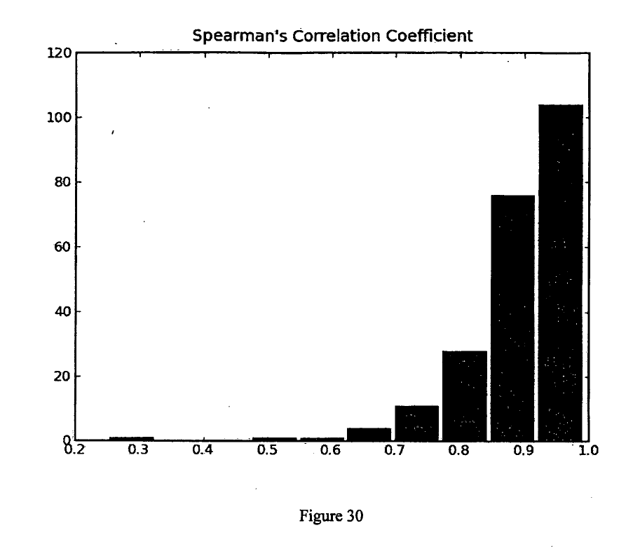

It is clear that five values of the coefficient are clumped at 0.8 and above while one is below 0.5. The low correlation coefficient corresponds to the derivative curve of the odd user in the multi-6 data set. Next the correlation coefficients for a set of 225 measurements were produced and compared to the correlation of the derivative curve of a different user as shown in FIG. 30. Note that in the graph of FIG. 30, the bin to the left with the single member that has a correlation coefficient near 0.3. This represents the odd user's data.

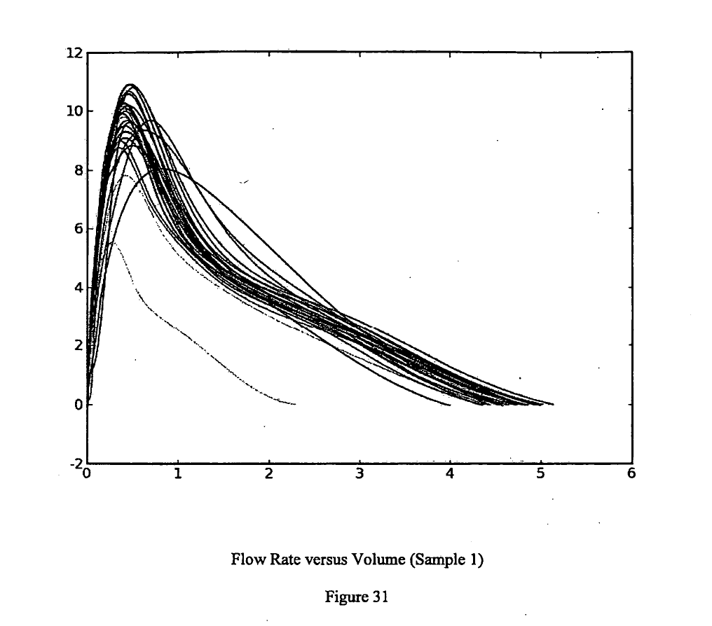

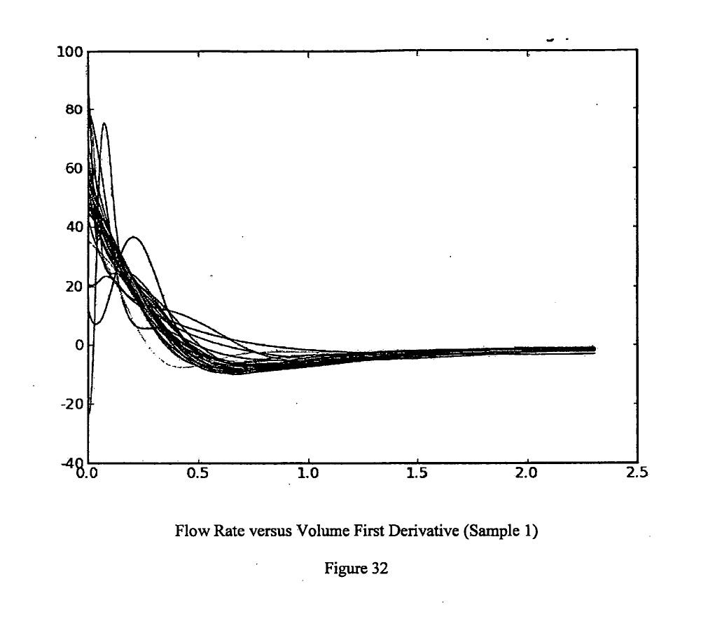

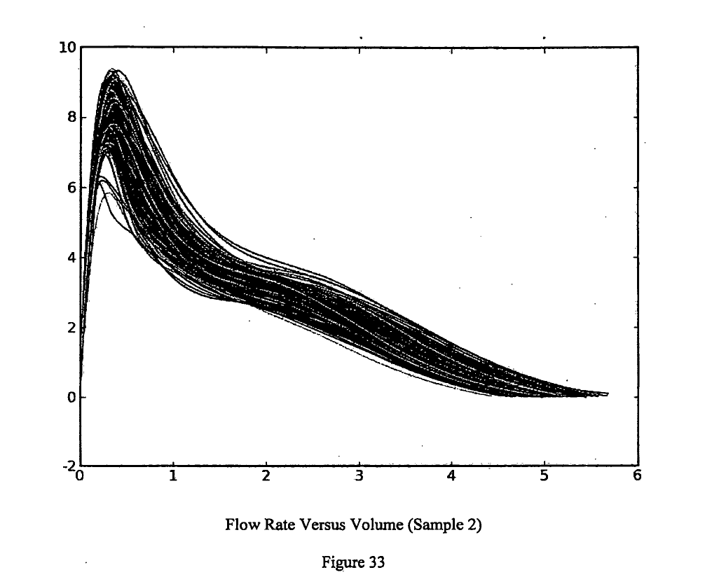

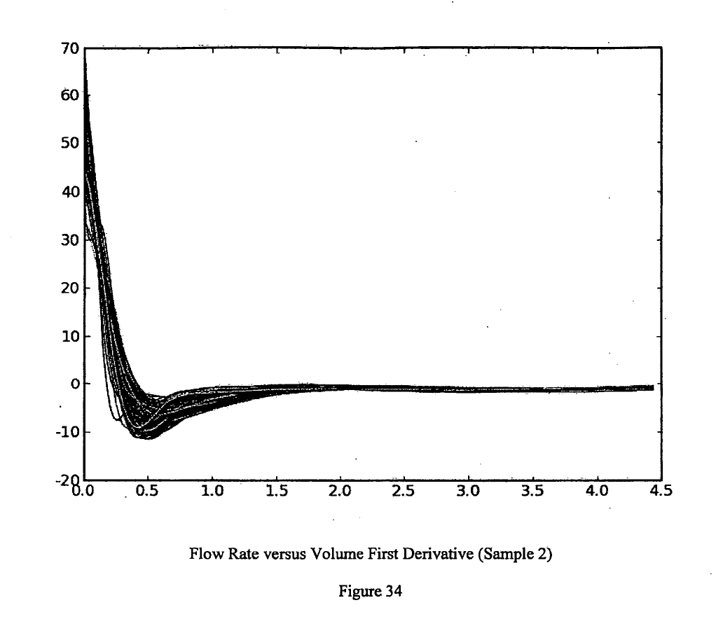

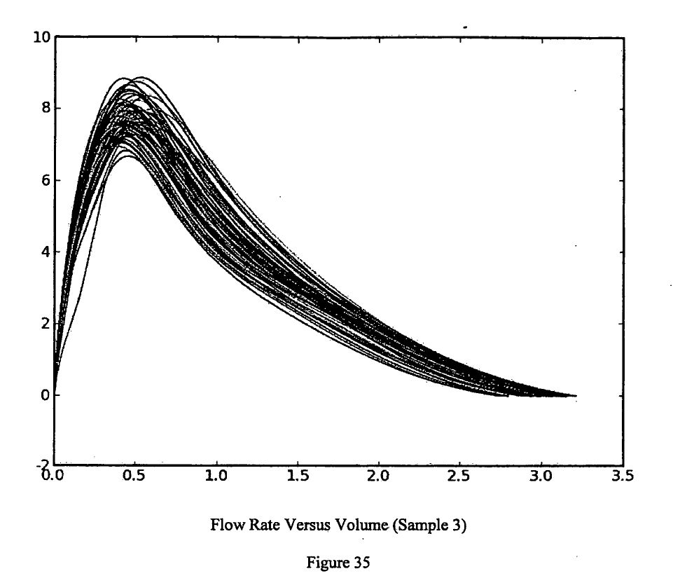

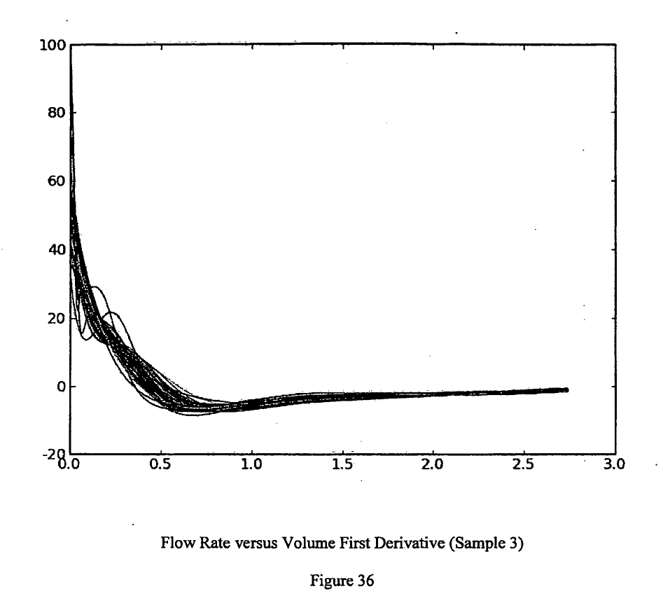

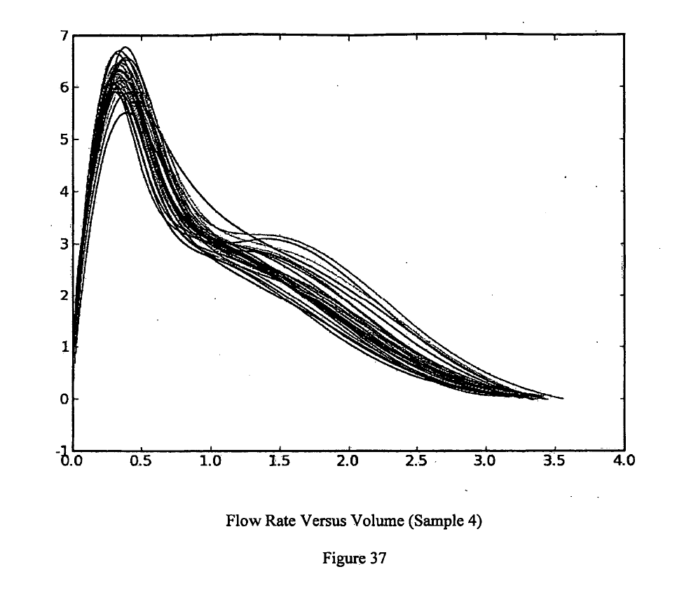

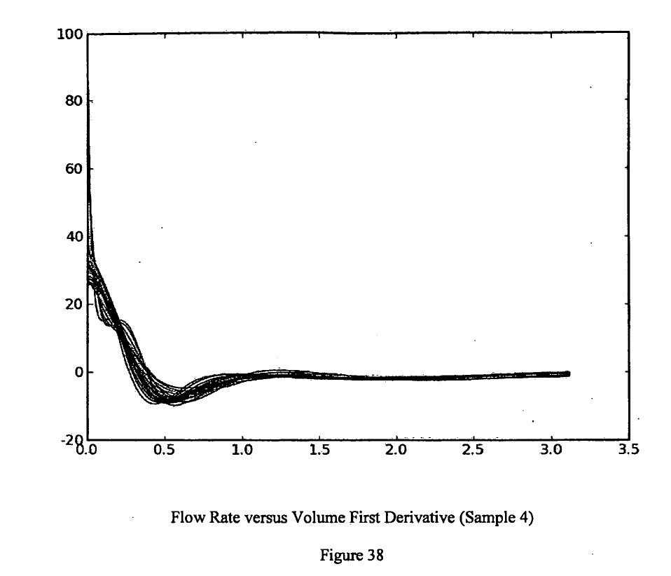

Next, span was run on four data sets of independent users and the serialized data (along with results of all calculations performed by span, such as fit, derivatives, correlation coefficients, data set size, and the like) were stored in individual repositories for future use. The repositories were named using user names that were embodied in the measurement data. For reference, Flow Rate vs Volume, Flow Rate vs Volume First Derivatives, for each of the data sets are shown in FIGS. 31-38.

Test of derivative correlation between full data sets was performed. Span was then modified to allow multiple data sets to be loaded simultaneously and for statistical correlation analysis of the derivative curves to be performed on them. As described herein, self-self correlation refers to correlation of the derivative curves from a single user against the average of the derivative curves for that user. Self-other correlation refers to correlation of the derivative curves from one user to the average of the derivative curves of another user. Also, “good” correlation is loosely used to mean values of statistical correlation coefficients clustered near 1.0. The term “poor” as used herein means not “good”. Note that the lengths (maximum volume) and peak flow rate of the curves within each user's data set and across multiple user data sets vary. The differing volume indicates different techniques may be used for calculating the statistical correlation.

The following three methods were derived and tested for usefulness. The first method includes selecting the shortest of the curves being analyzed and the average curve and only uses points in that region of each curve. The second method includes selecting the ½ maximum volume point of a curve being analyzed and statistically correlate between zero volume and that point (attempt to eliminate much of the linear region). The third method includes selecting the longest of the curves being analyzed and the average curve and uses the length of the longest to determine the range across which the analysis is performed. Extrapolate the shorter of the two curves so the two have the same length.

Each of these methods was implemented and tested and it was noted that in all cases, self-self statistical correlation was good. No single technique always produced self-other statistical correlation coefficients that were always poor. However one or more of the methods would provide self-other coefficients that were poorer than the others, so all three methods are always executed and results compared. The one that provides the poorest statistical correlation is chosen. Having done this, it was evident that in some cases, self-other correlation was not satisfactorily poor. The ideal analysis provides for the distribution of self-self correlation coefficients to have no overlap with the distribution of self-other coefficients.

Accordingly, the following modifications of the span prototype were implemented and tested. First, a minimum value of the correlation coefficient can be defined and when data is loaded for analysis (or, as implemented, selected for storage in the repository for future use) any curve that has a self-self correlation less than the minimum value is pruned from the set of curves.

Second, since it was noted that the set of flow rate versus volume curves from a single user tended to have the same maximum volume and peak values, two de-rating factors were defined and applied to the coefficient calculation:

volume de-rating factor=1.0−{abs(totalVolume[i]−averageTotalVolume)/totalVolume[i]}; and 2.1.

peak de-rating factor=1.0−{abs(peakFlowRate[i]−averagePeakFlowRate)/peakFlowRate[i]}. 2.2.

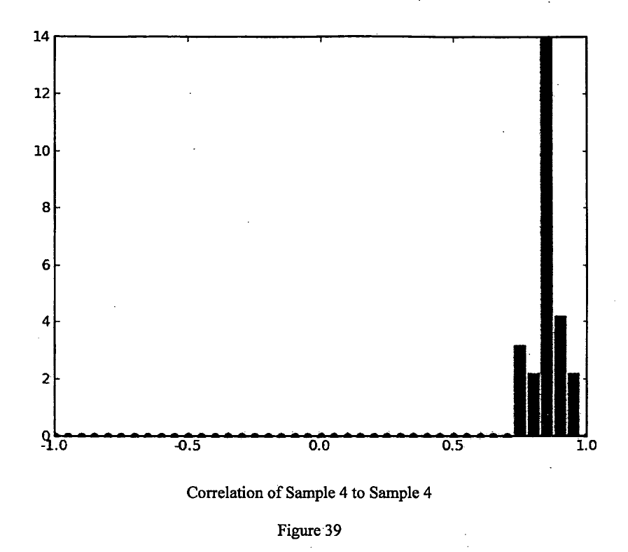

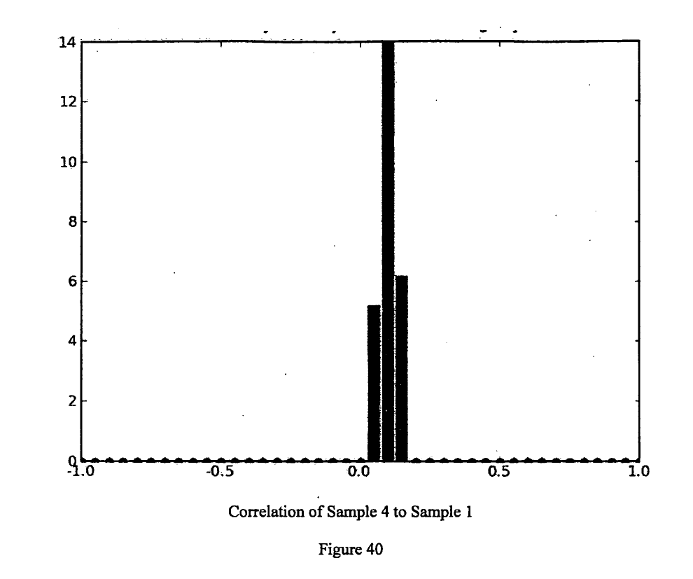

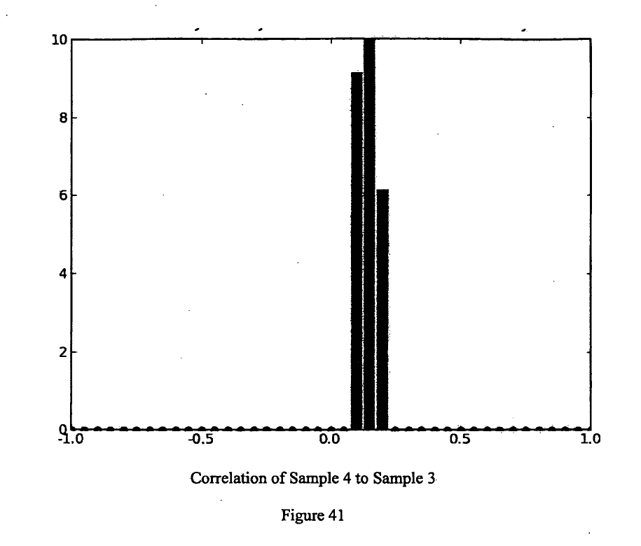

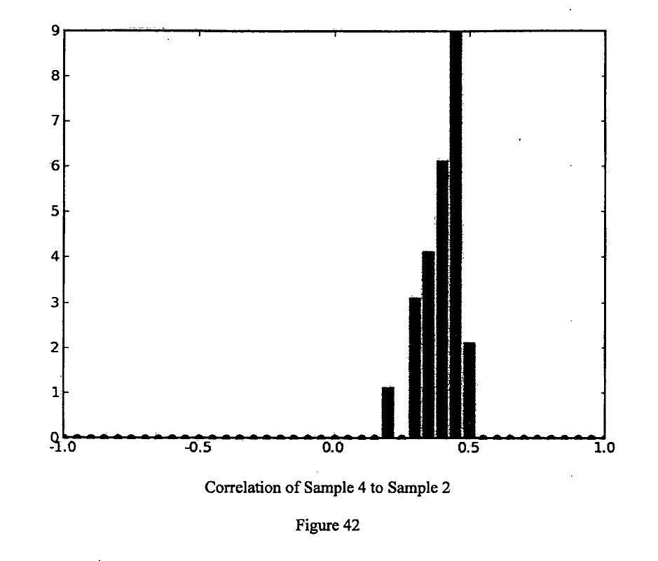

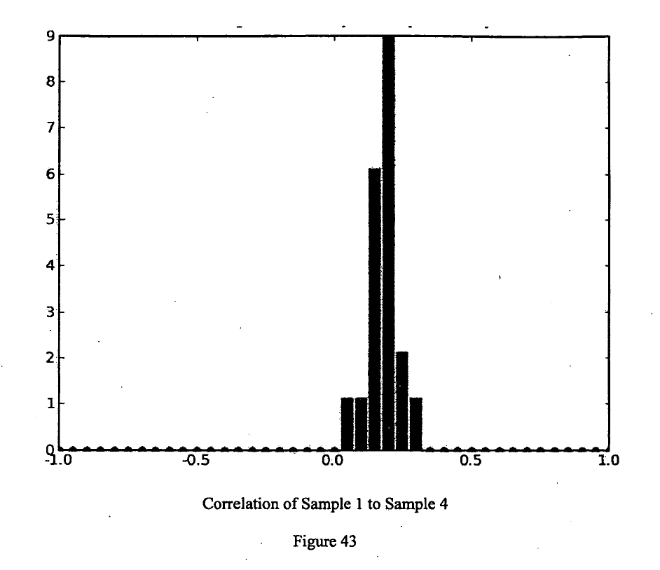

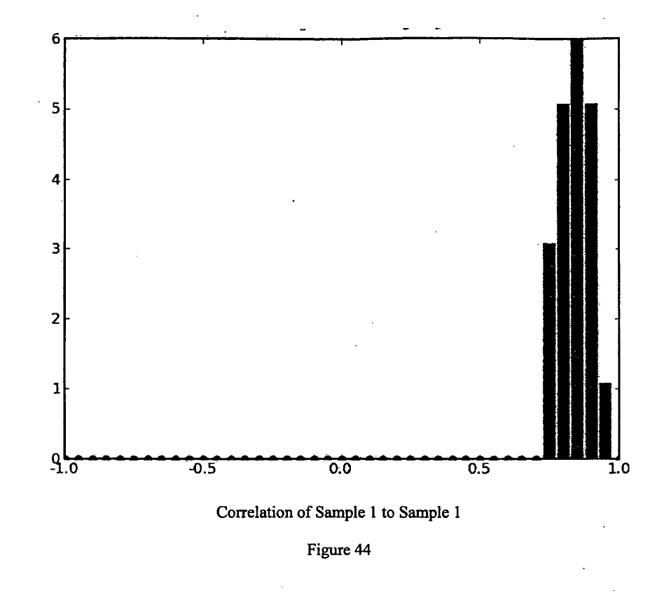

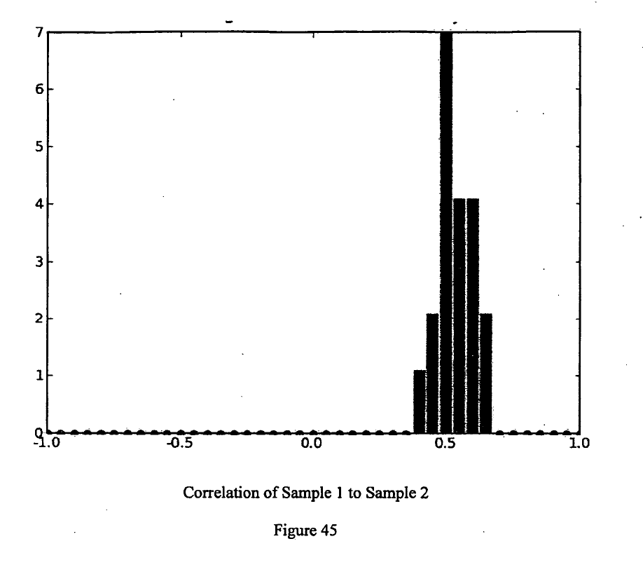

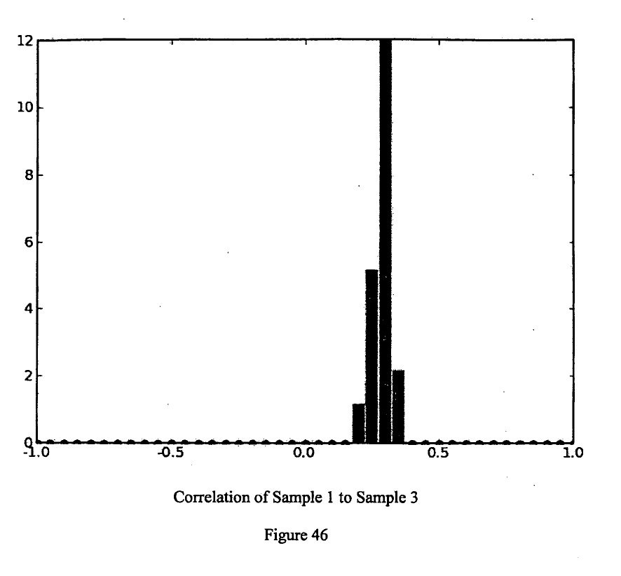

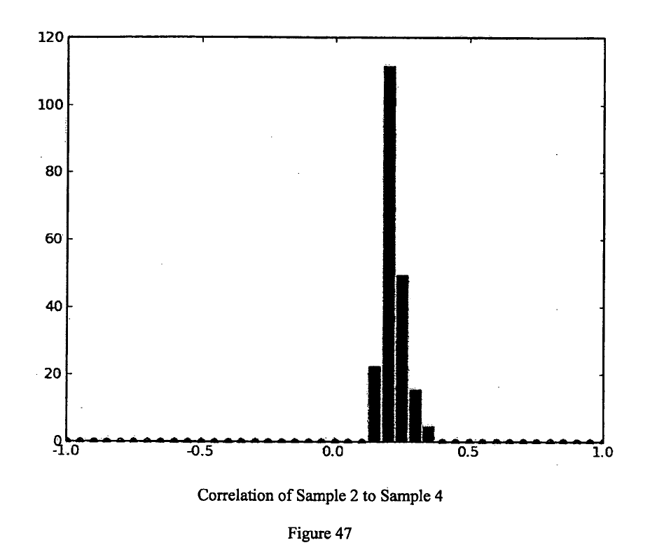

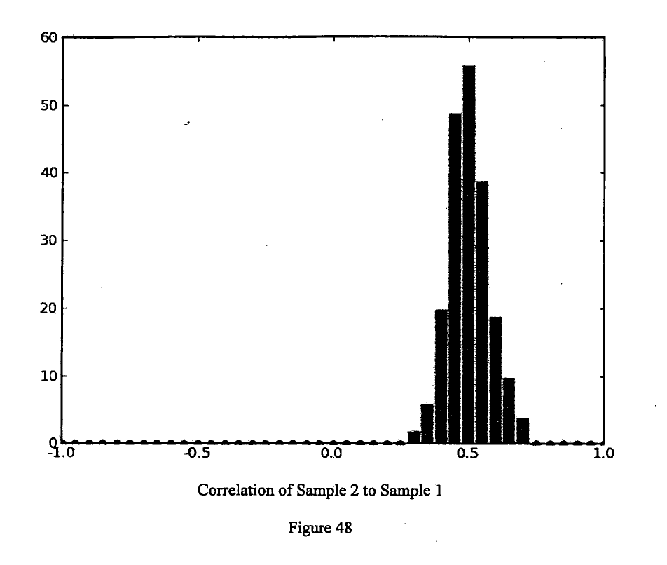

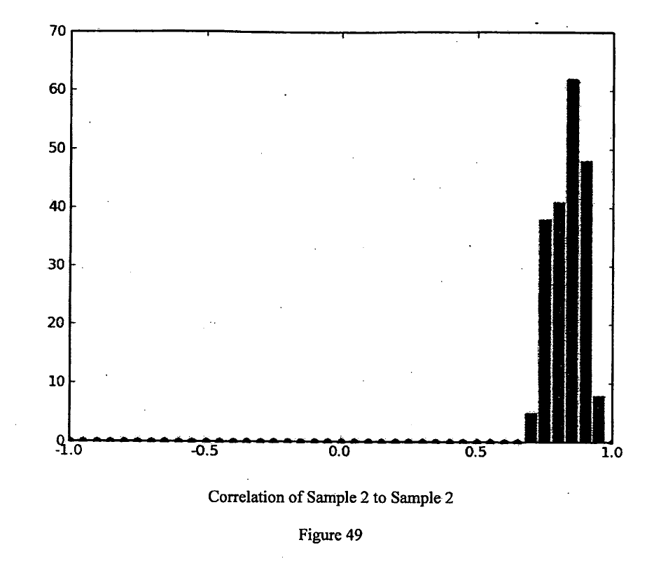

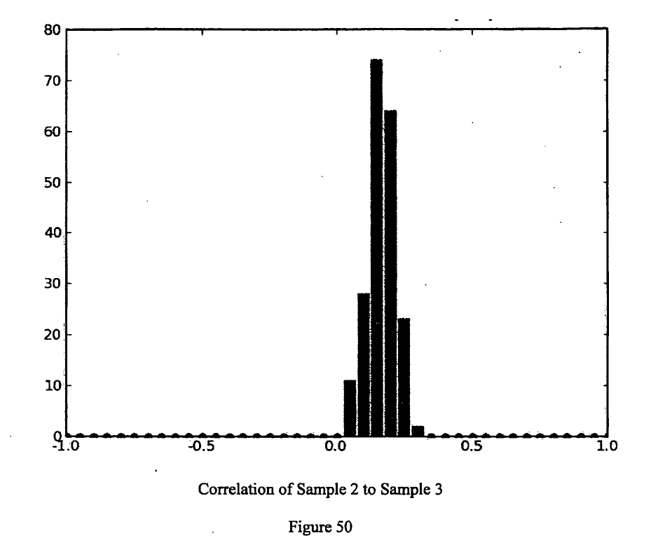

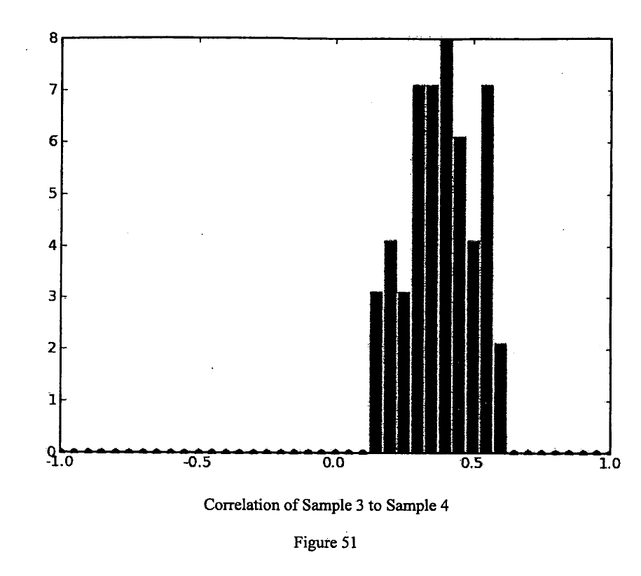

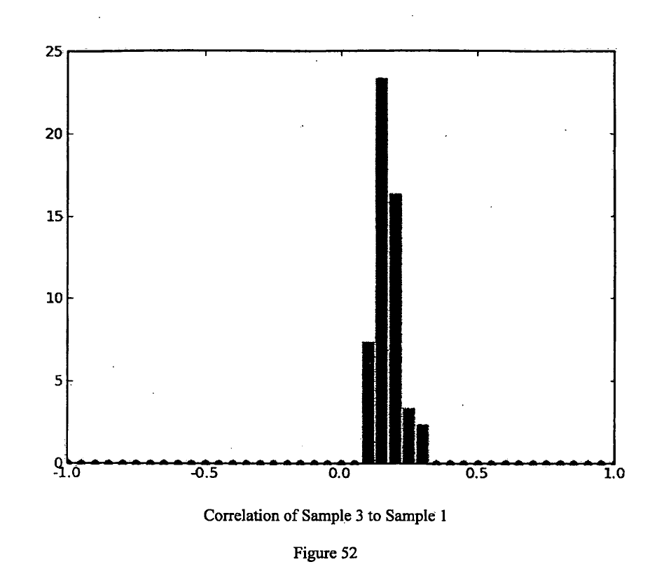

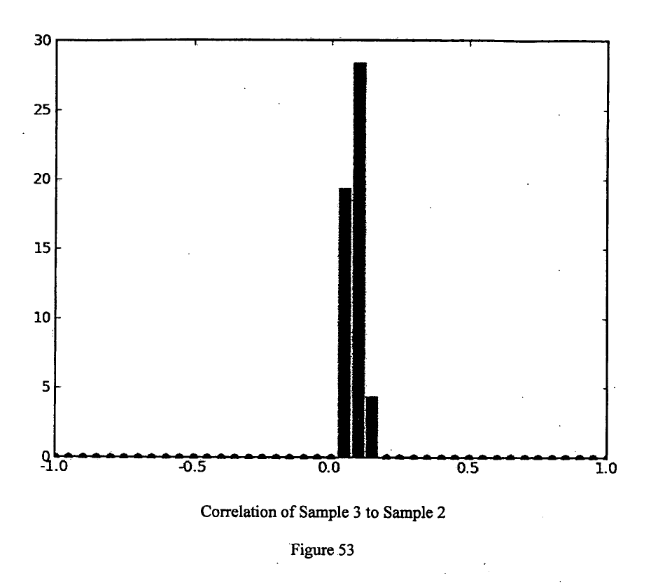

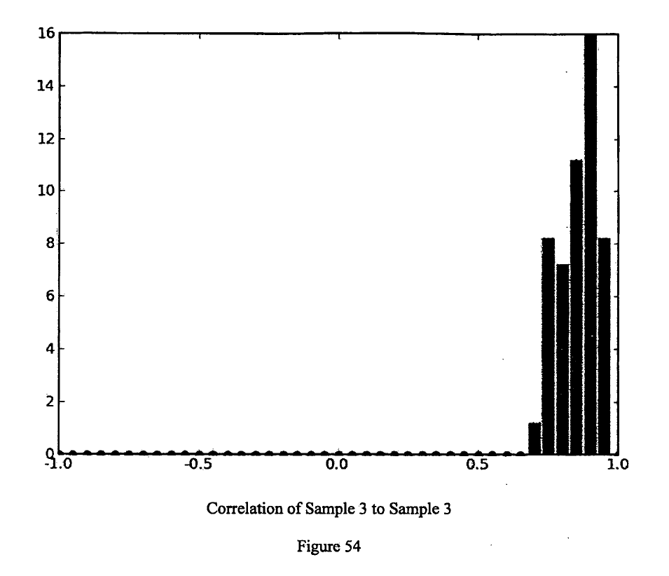

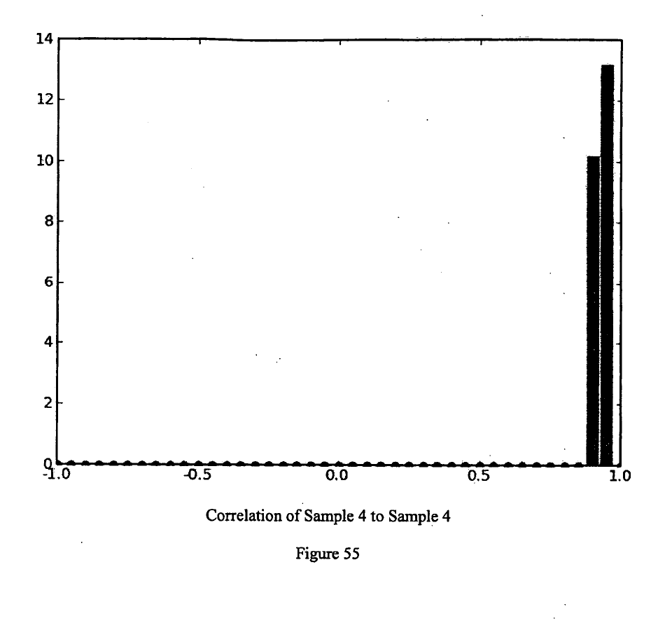

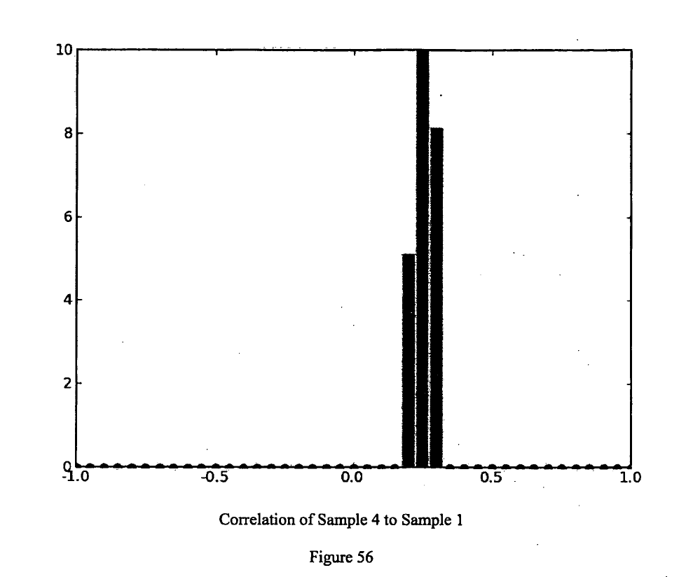

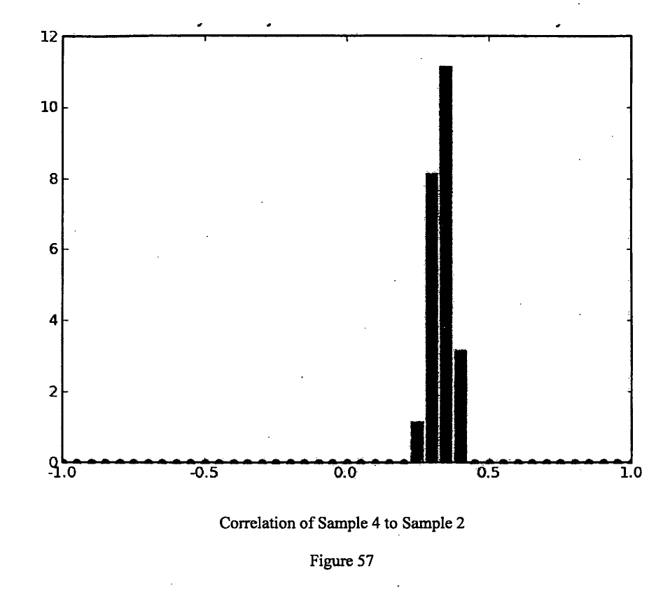

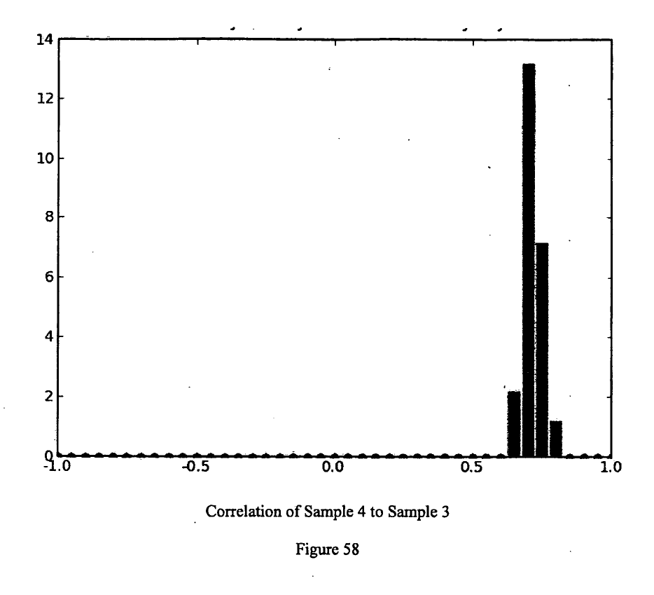

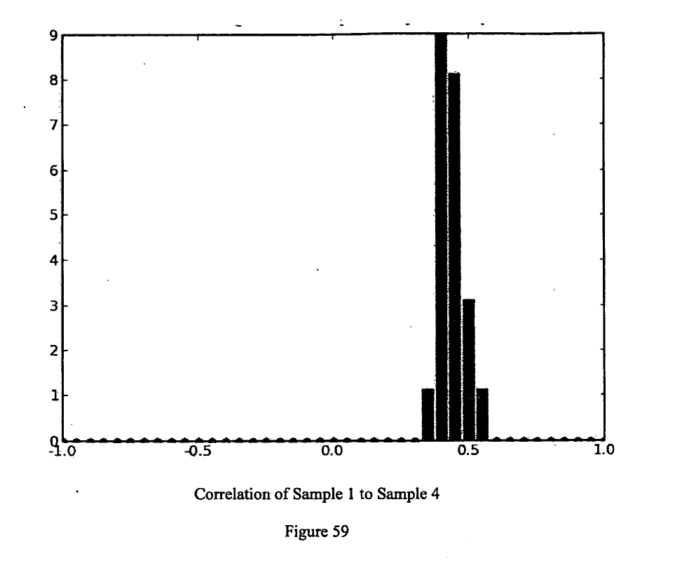

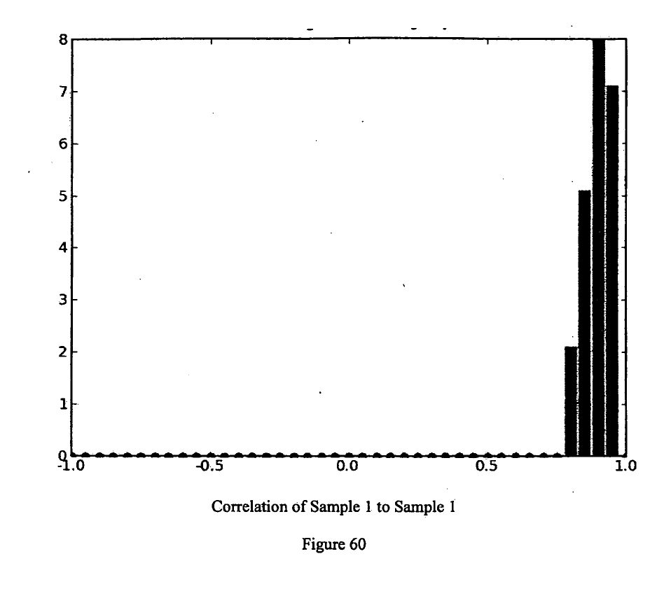

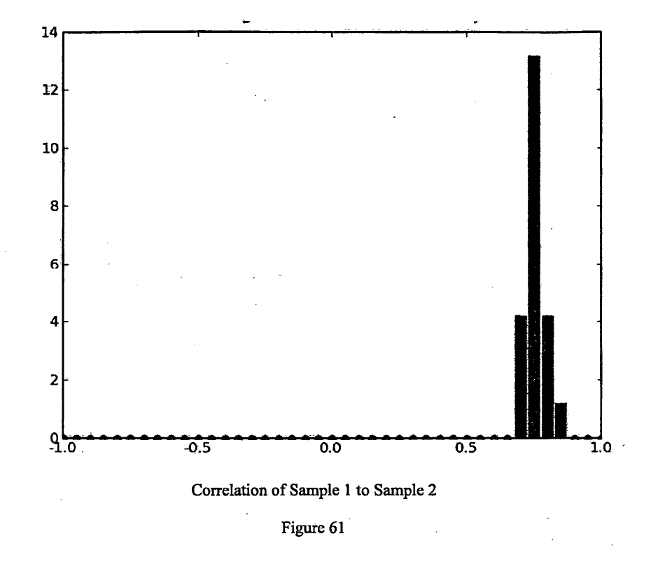

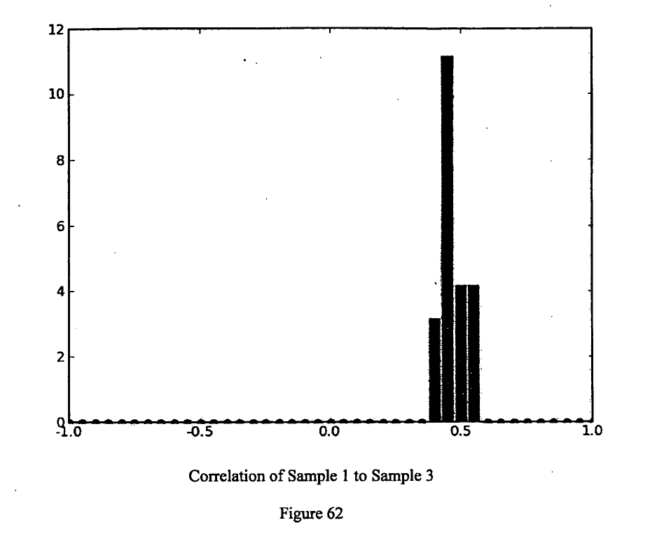

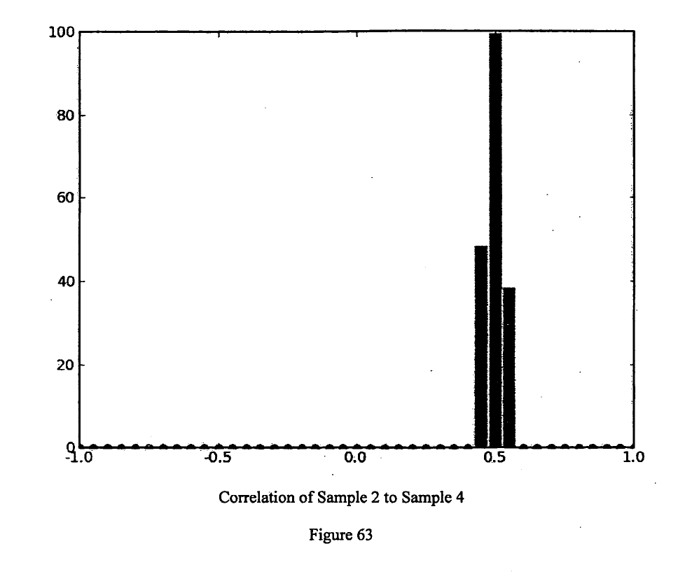

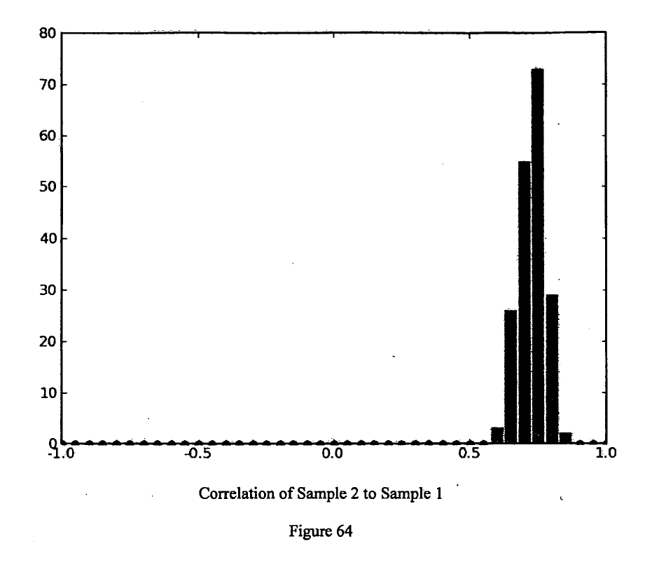

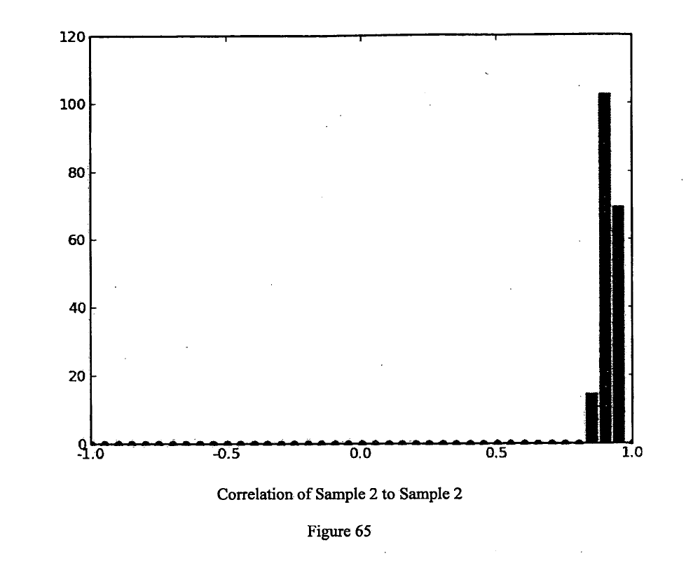

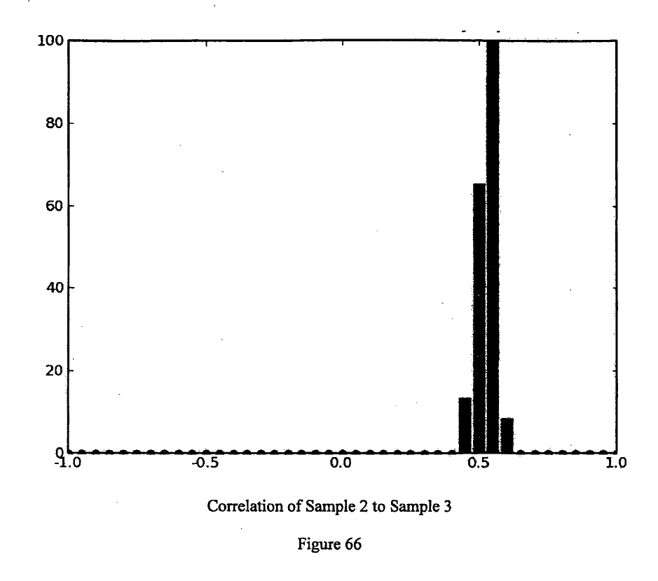

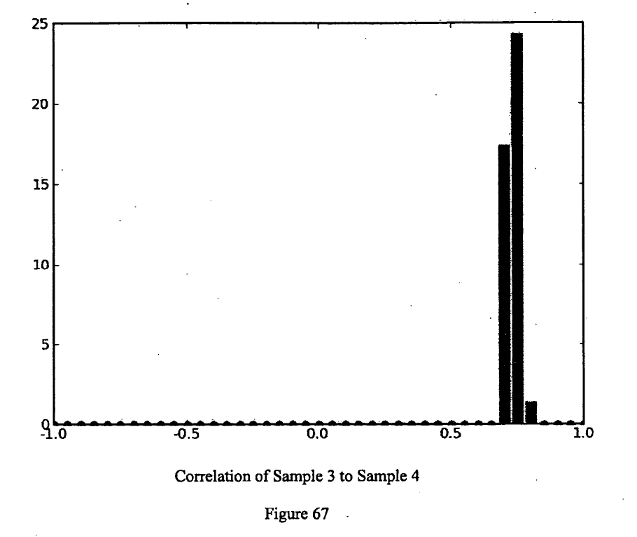

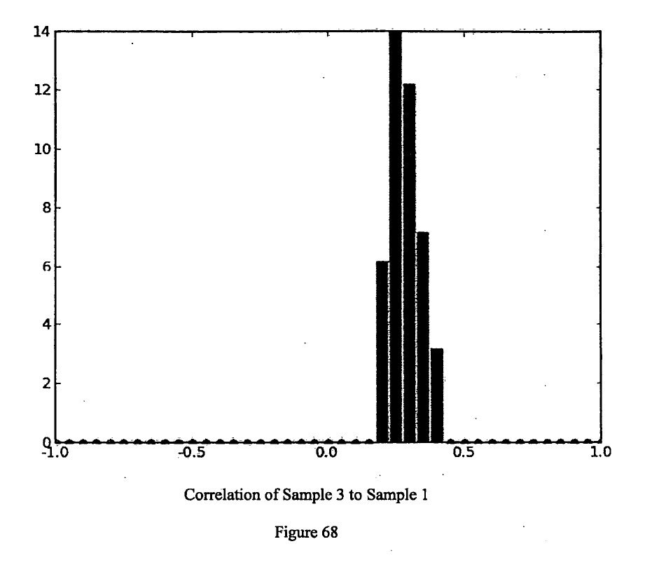

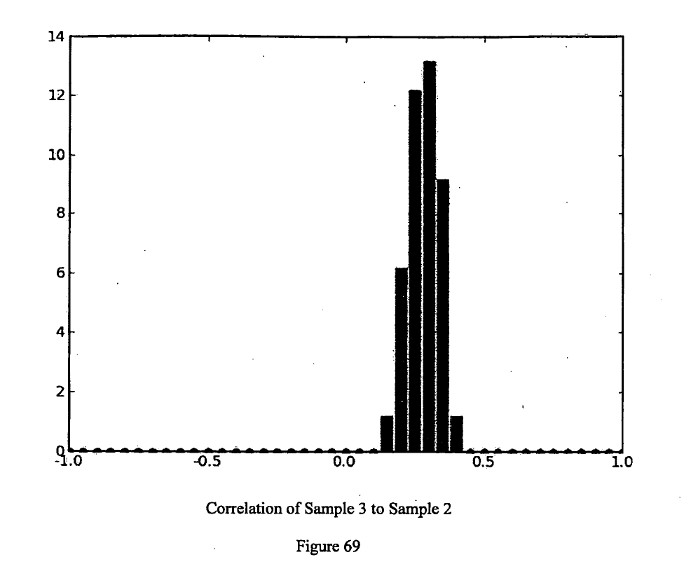

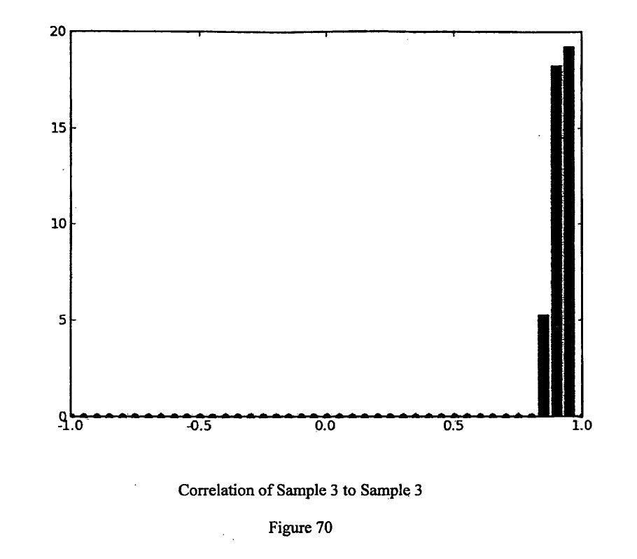

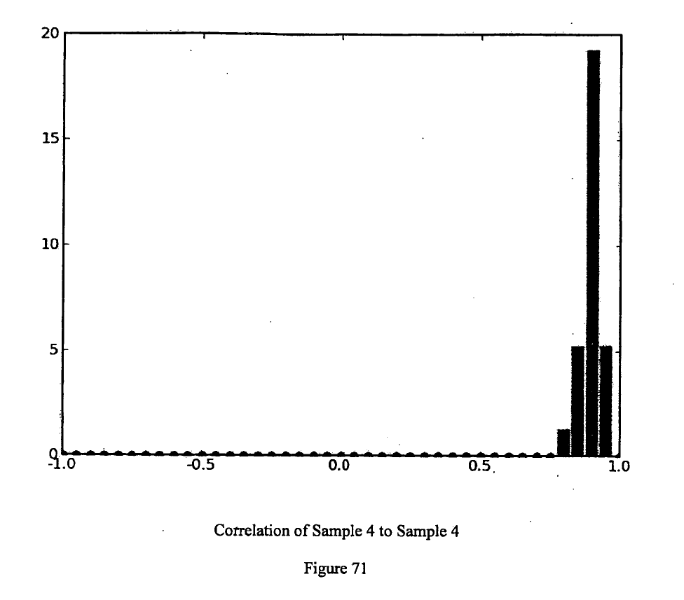

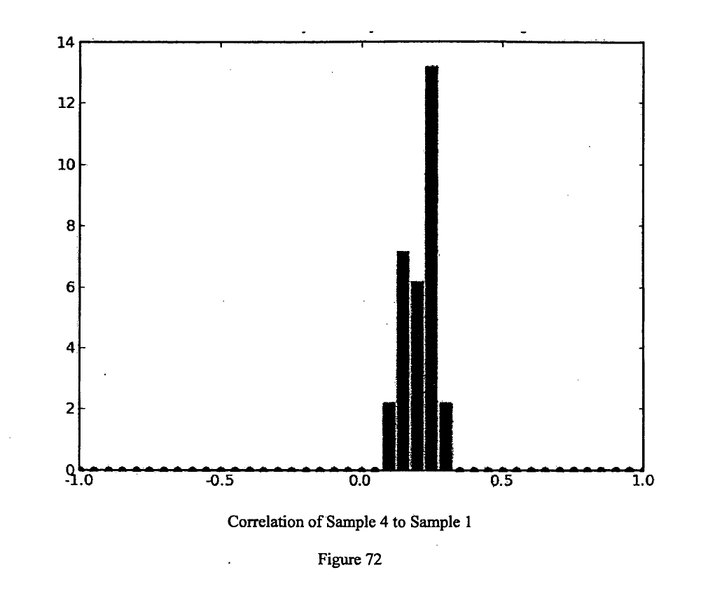

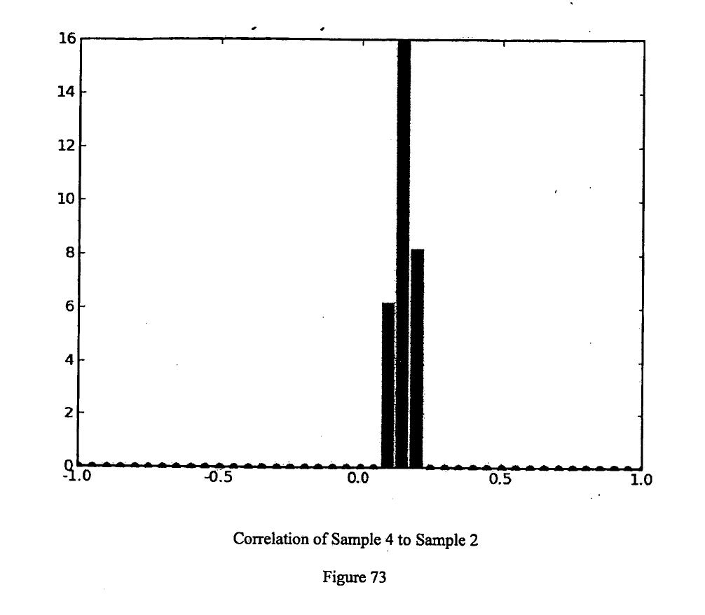

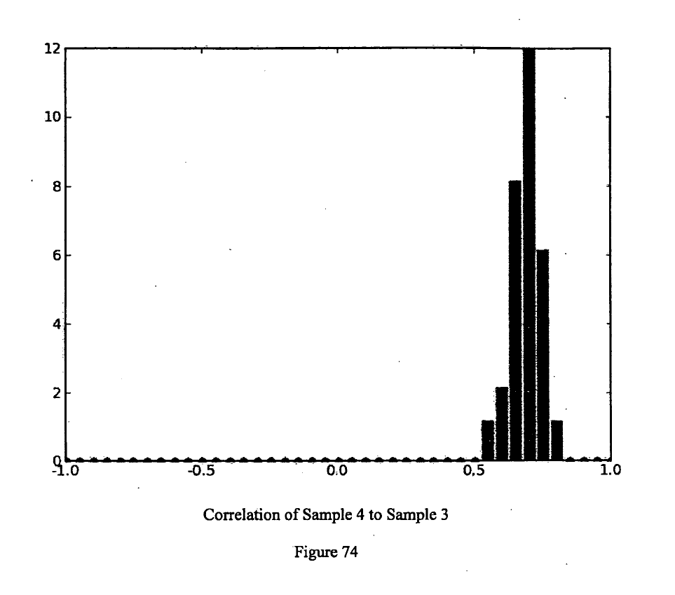

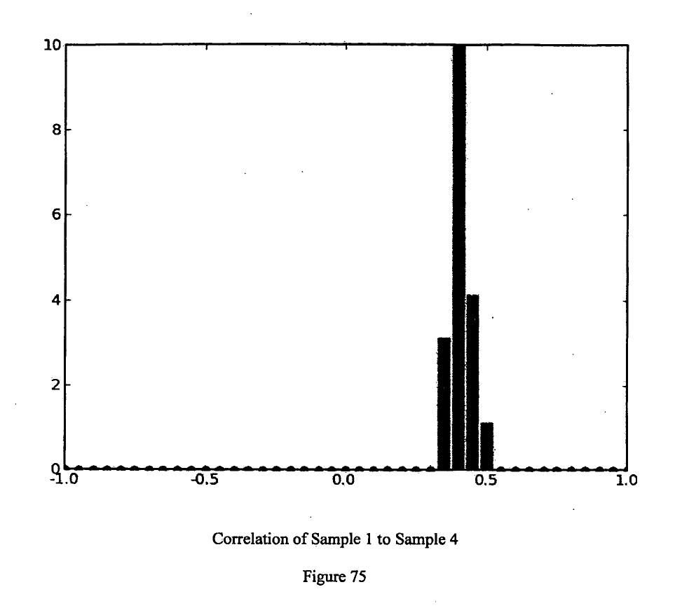

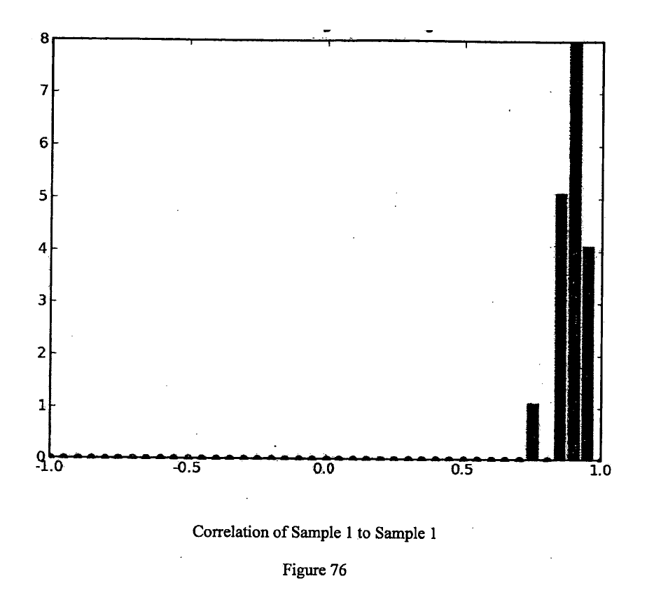

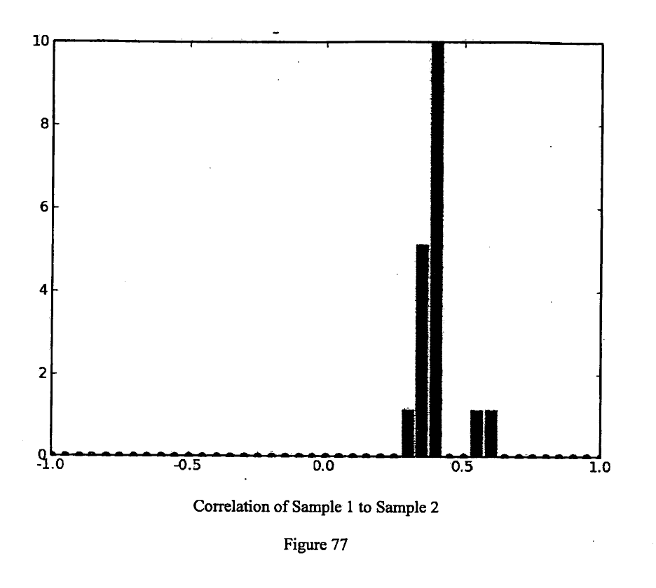

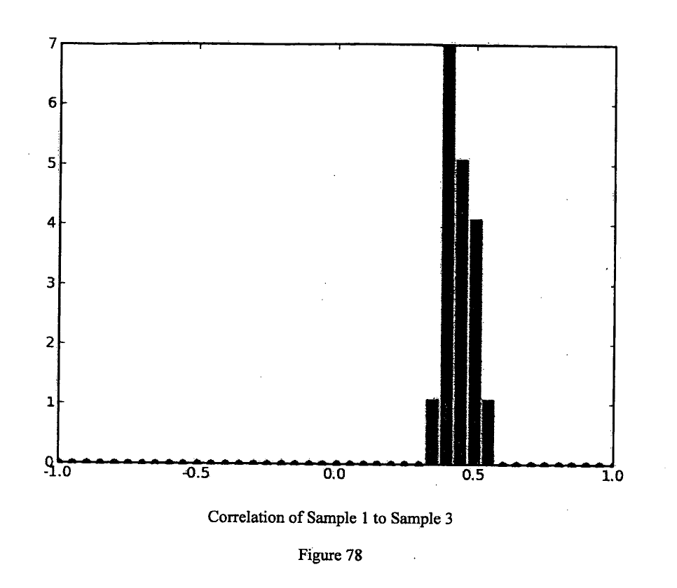

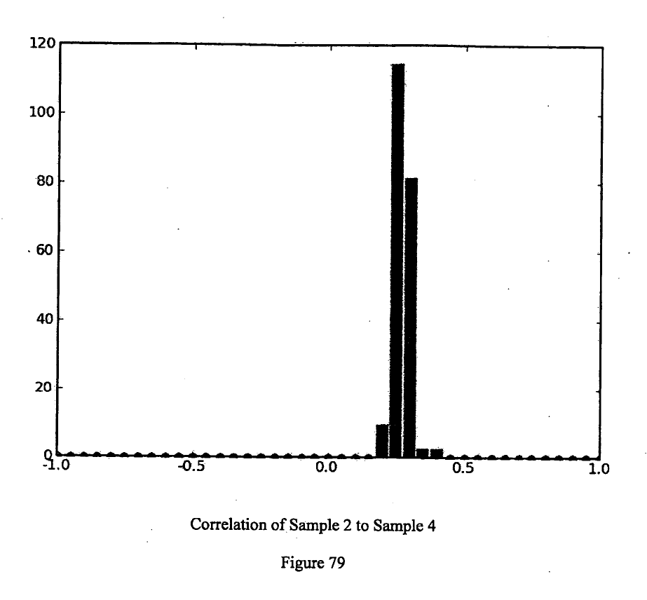

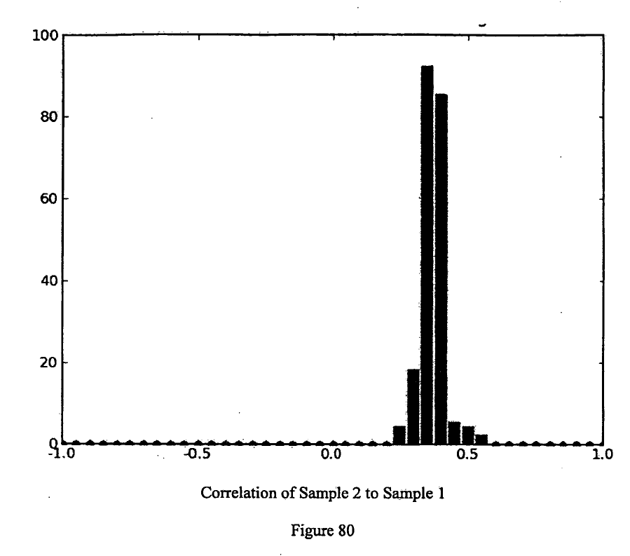

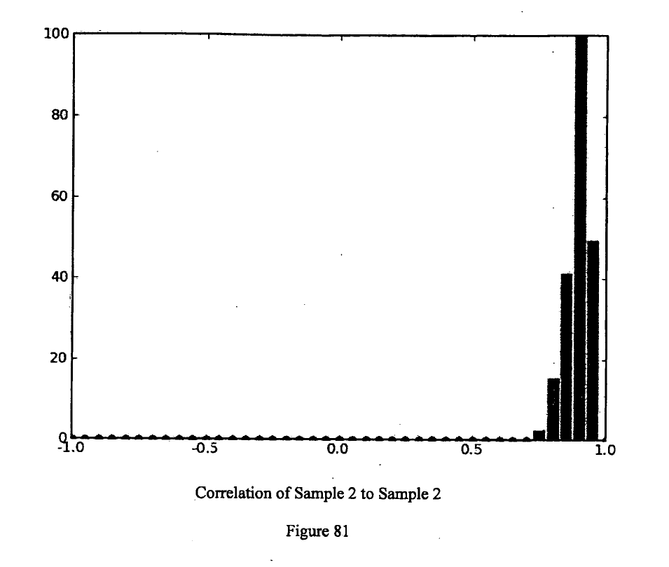

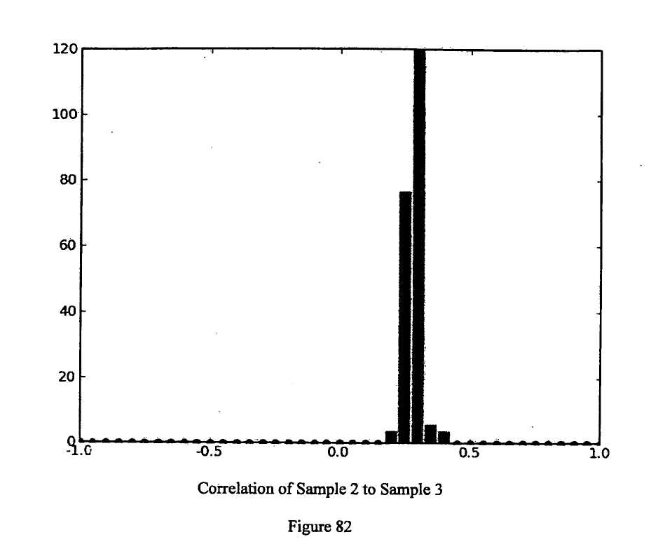

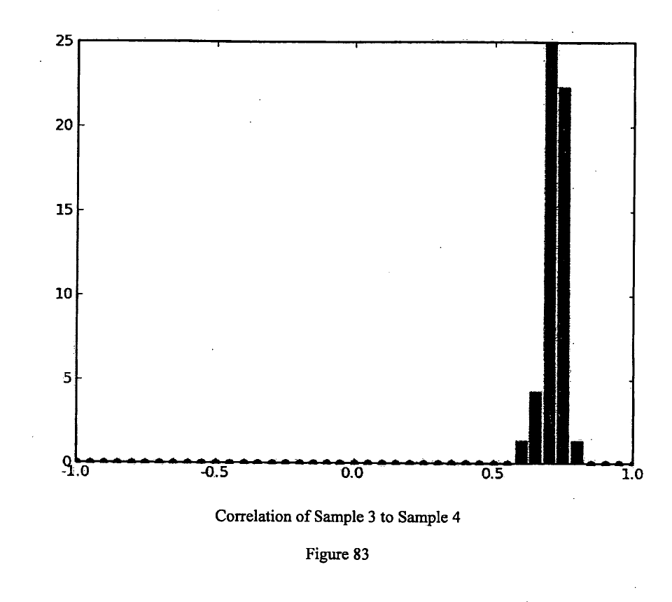

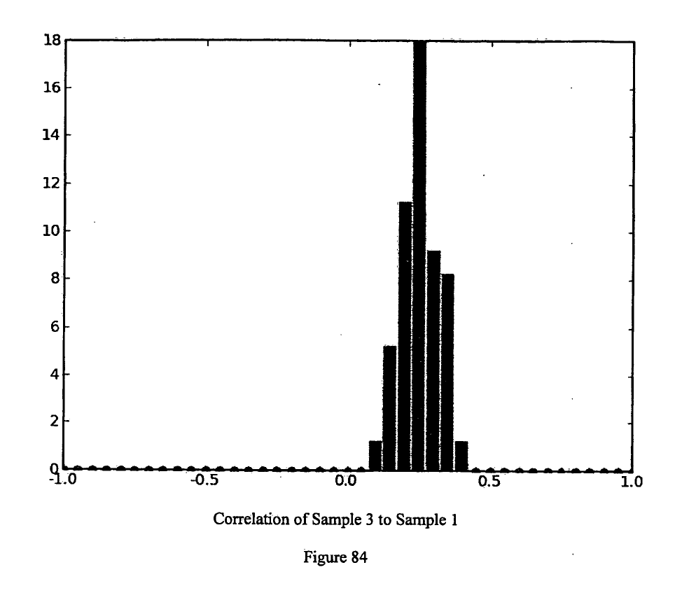

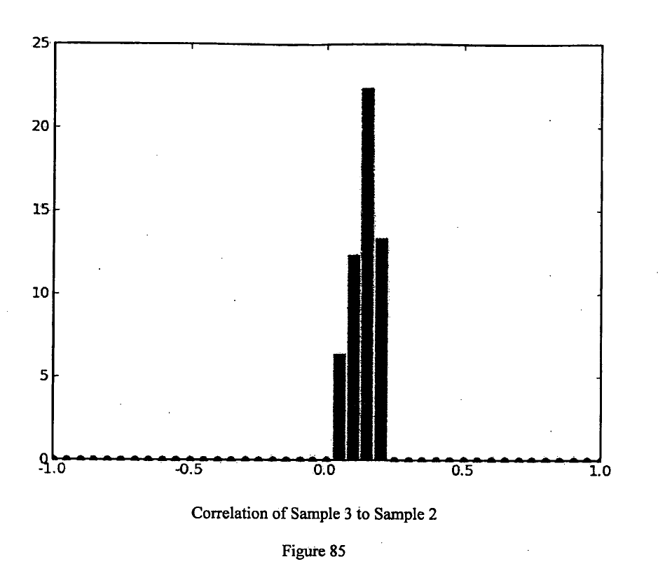

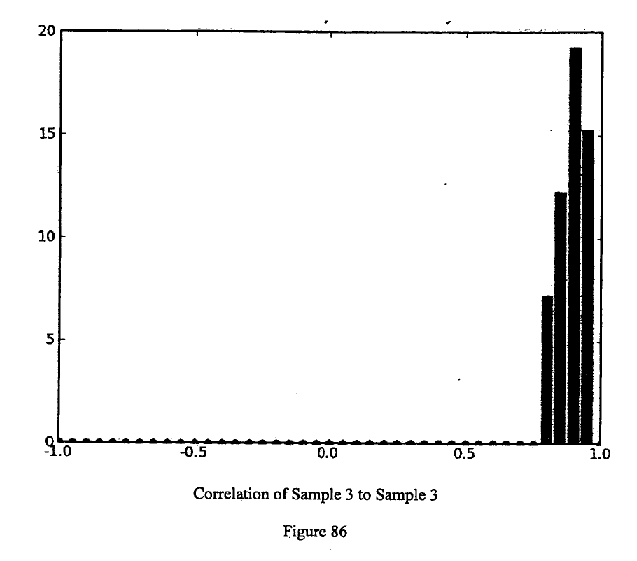

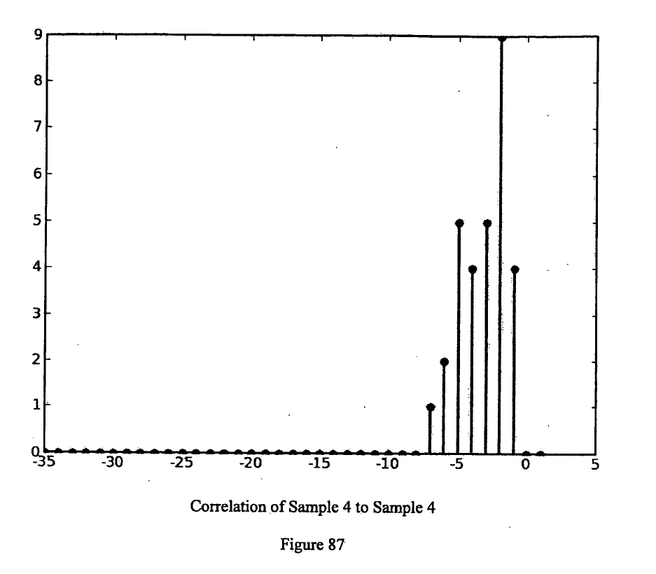

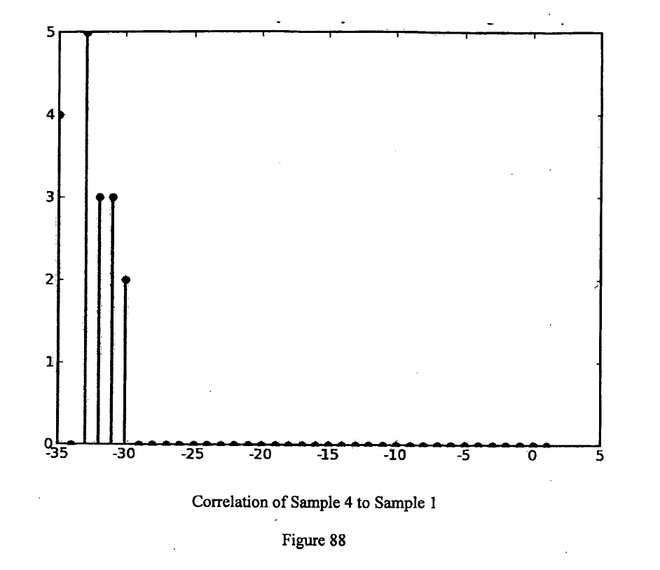

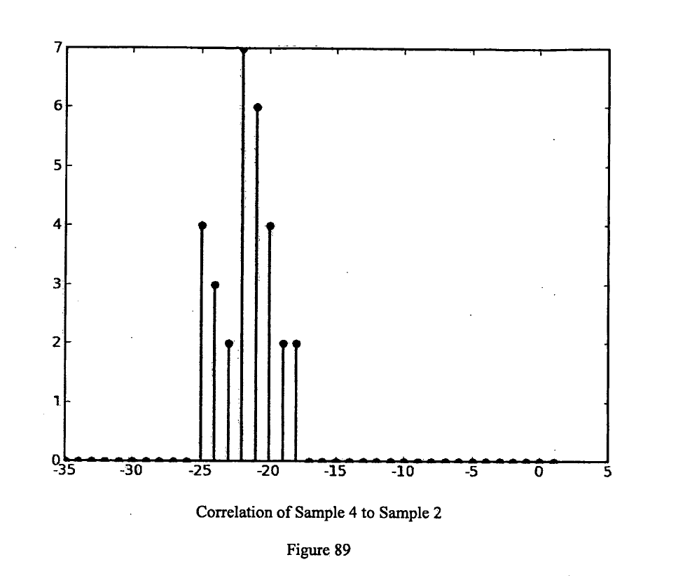

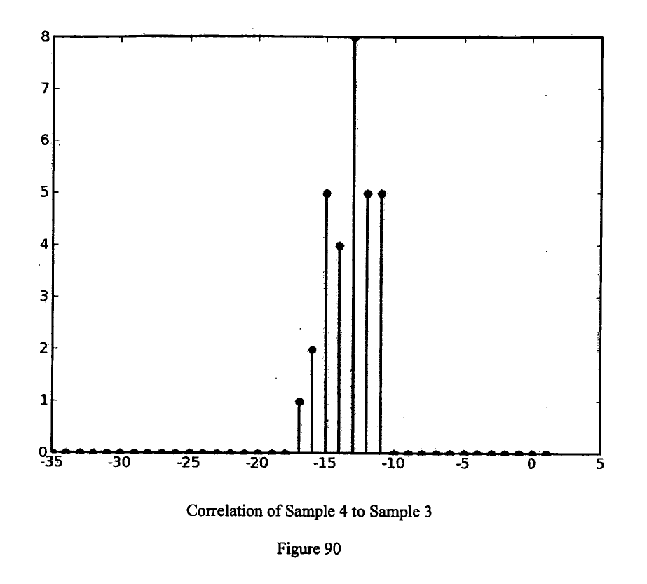

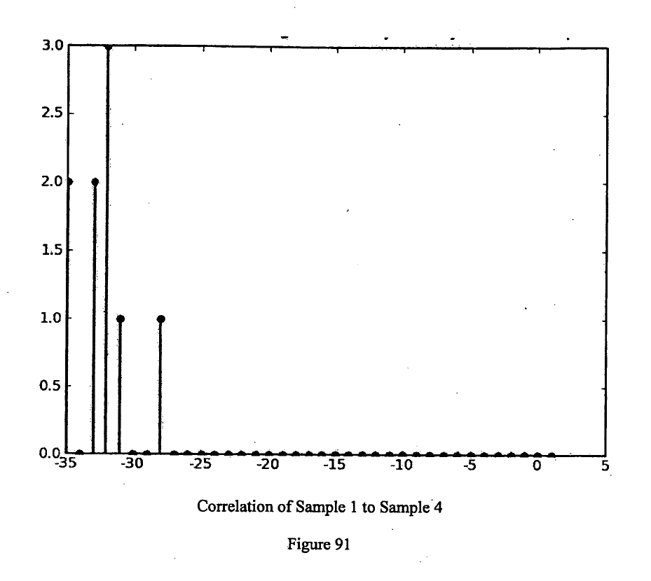

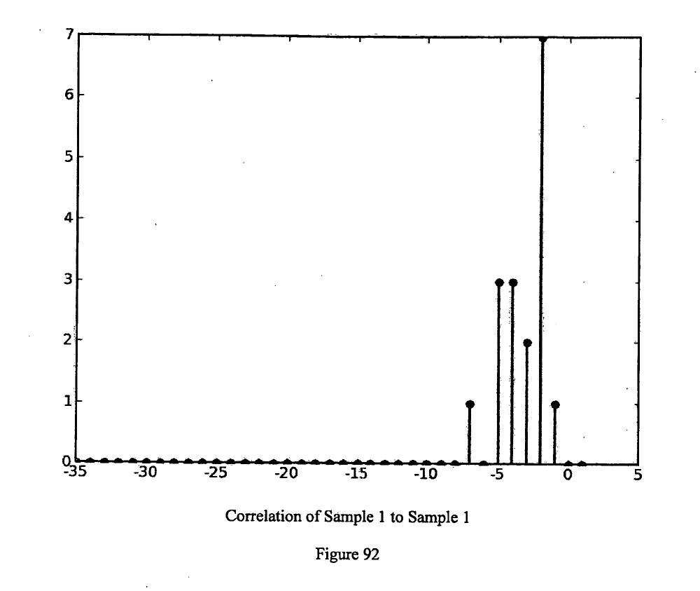

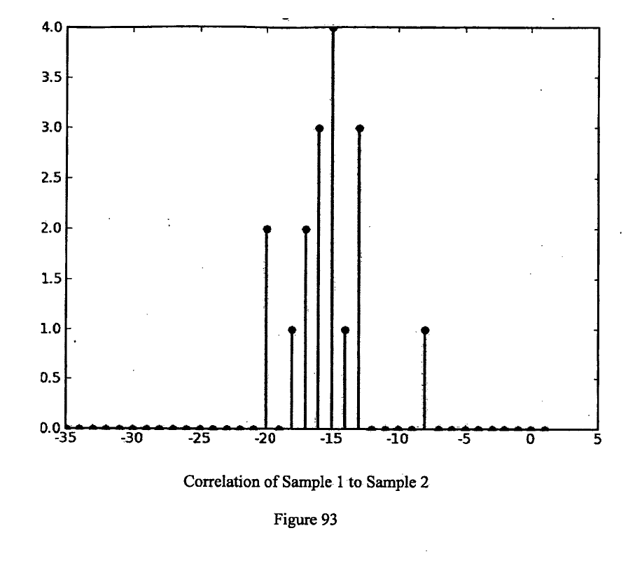

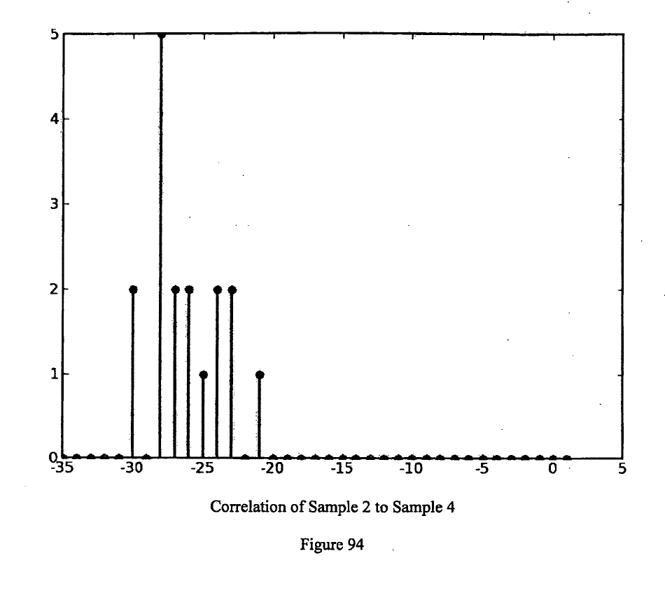

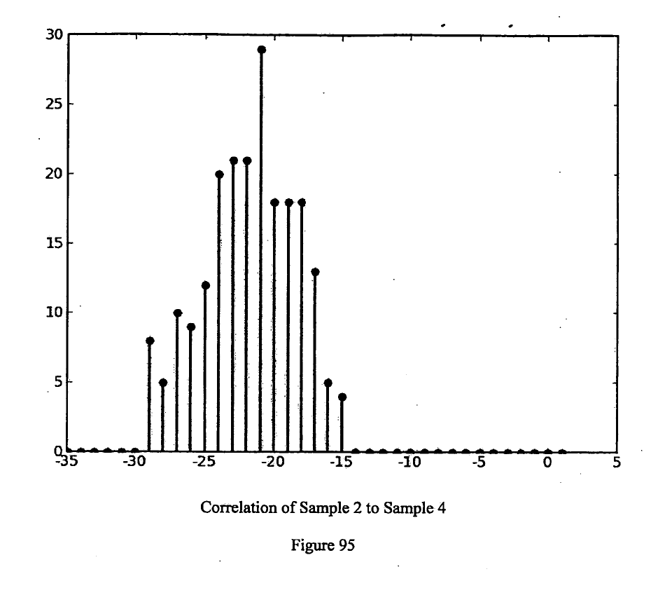

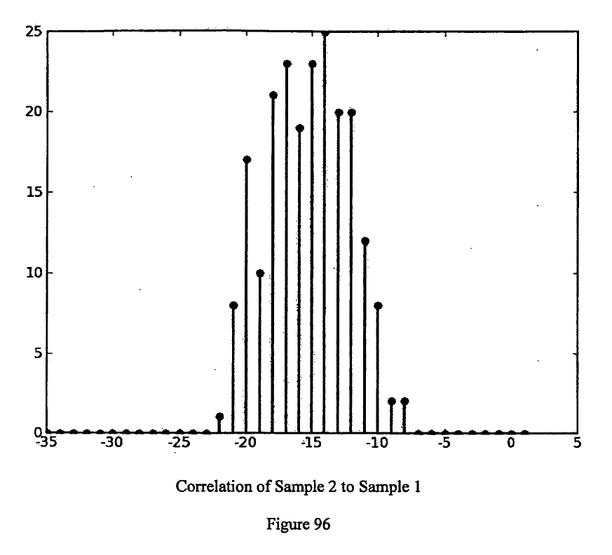

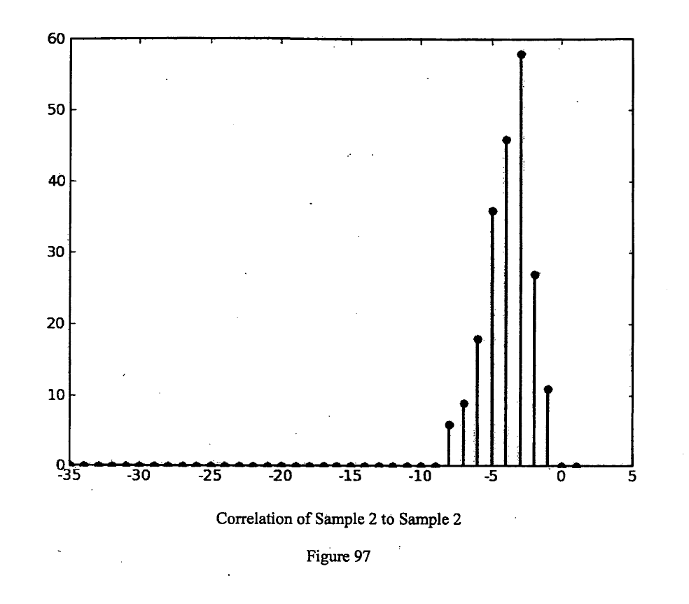

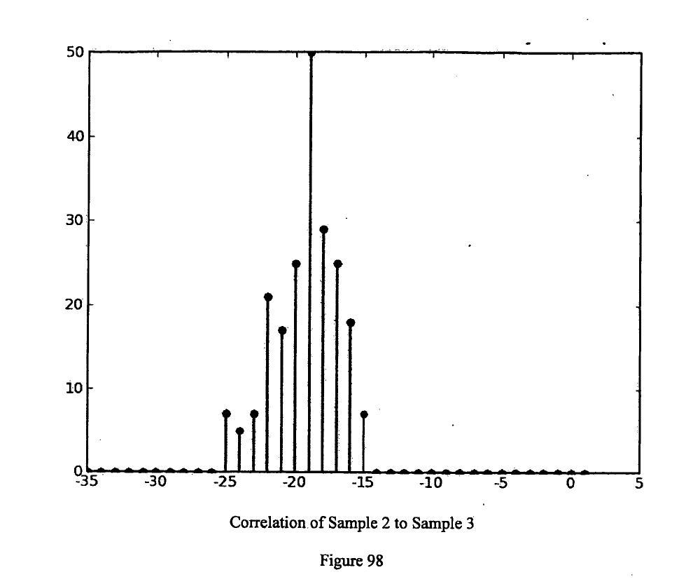

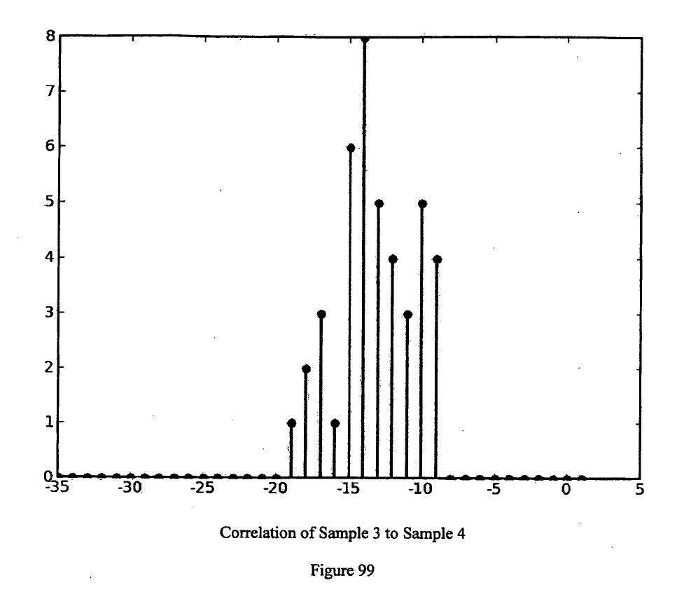

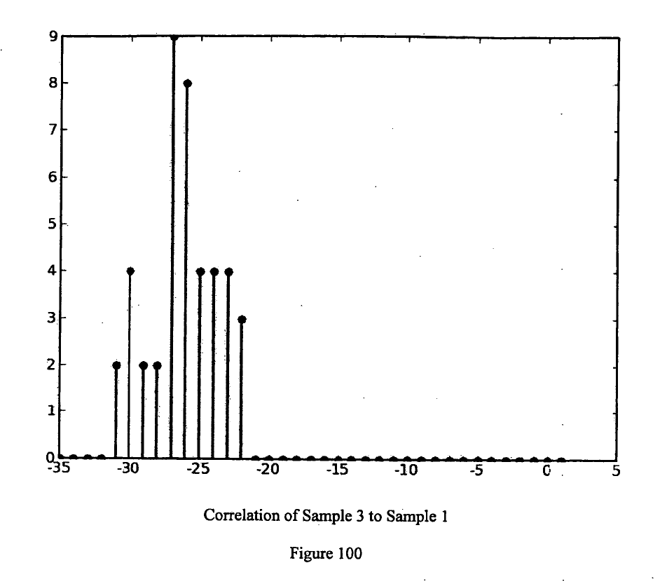

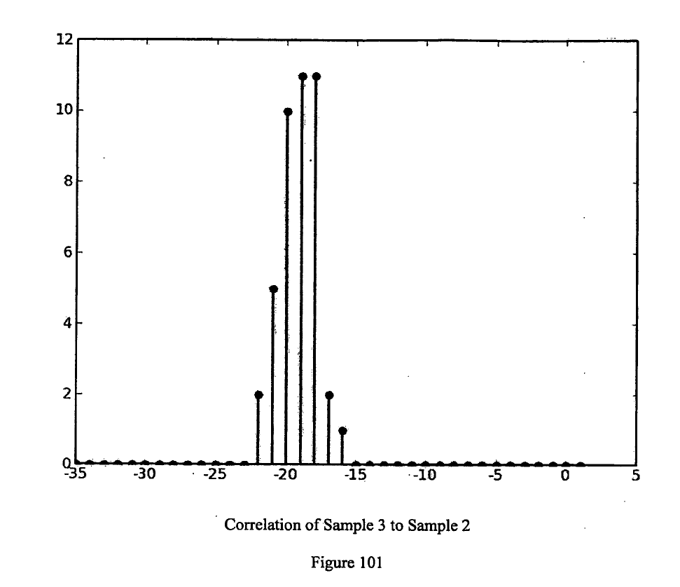

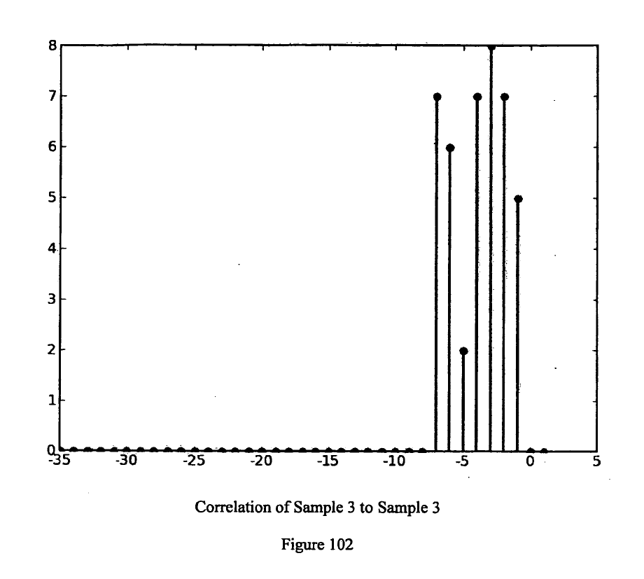

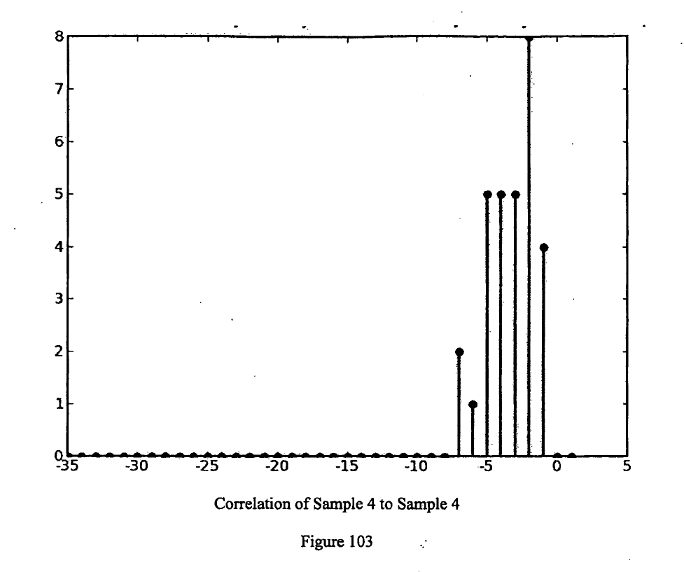

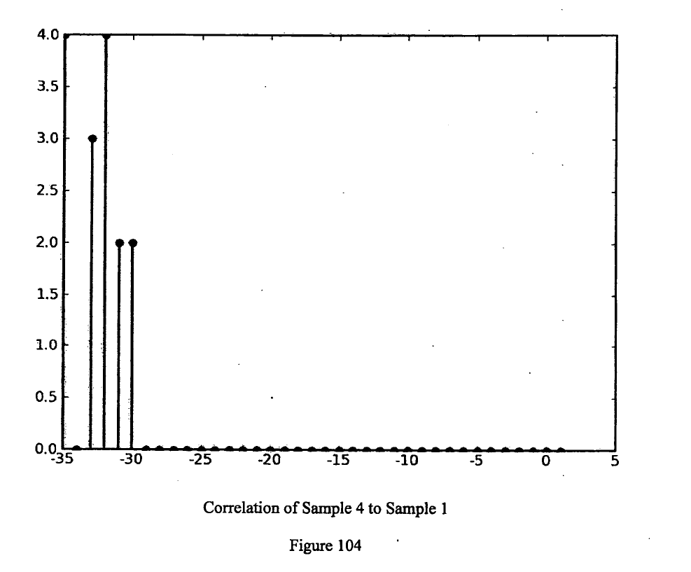

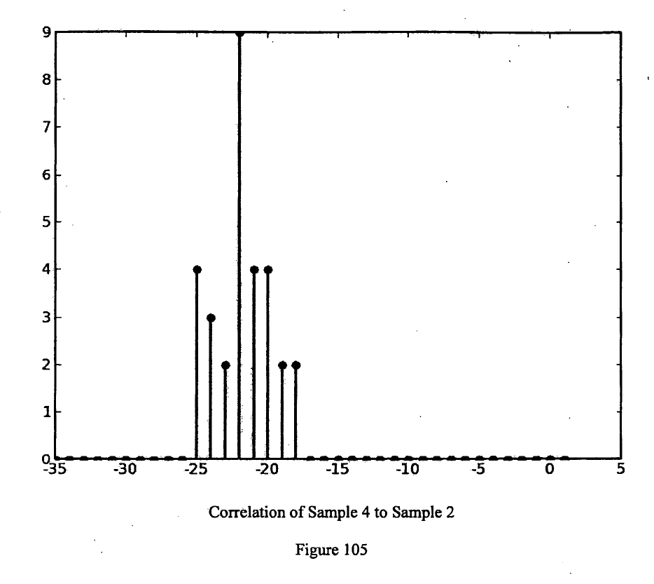

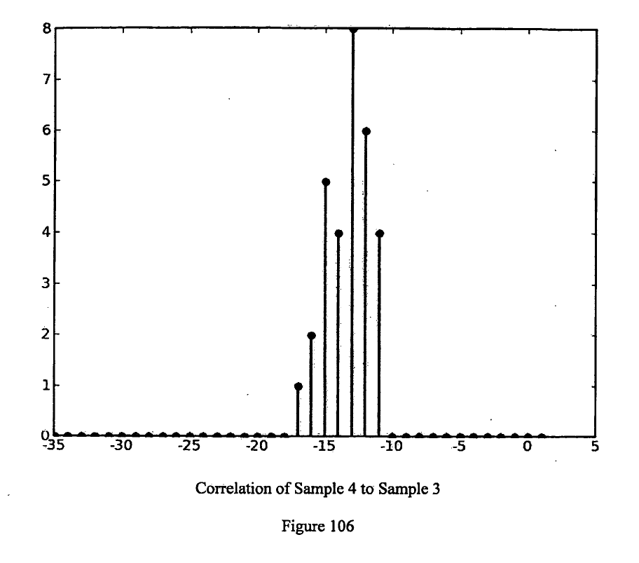

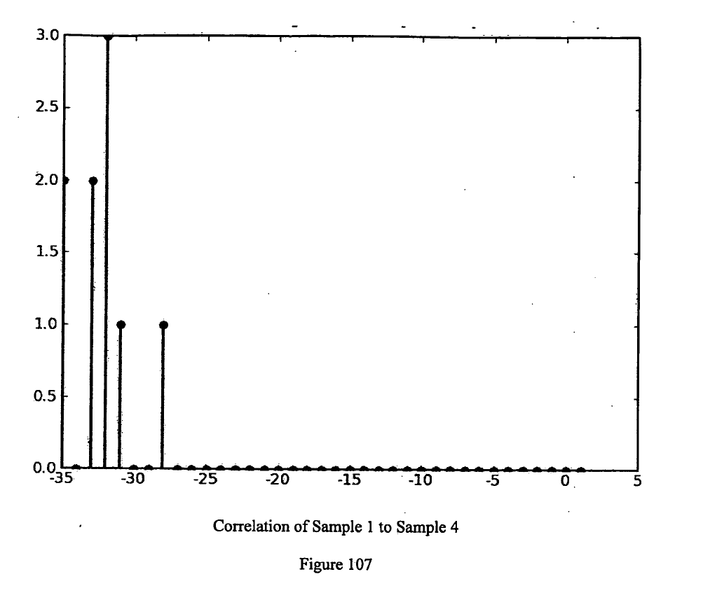

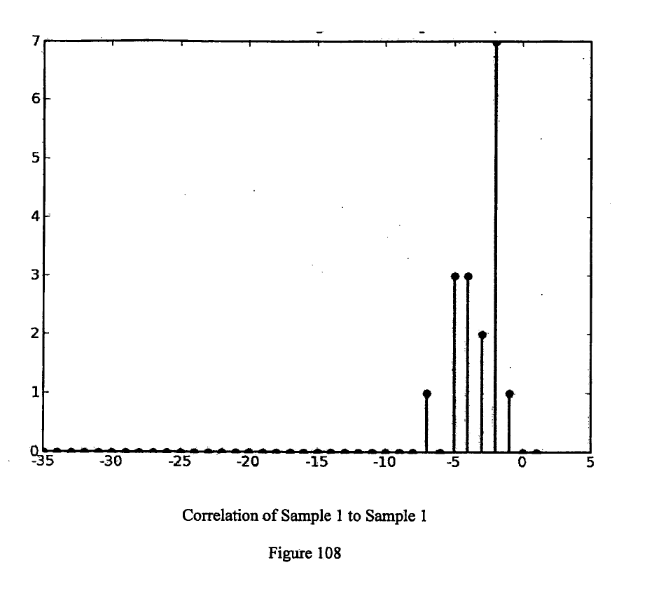

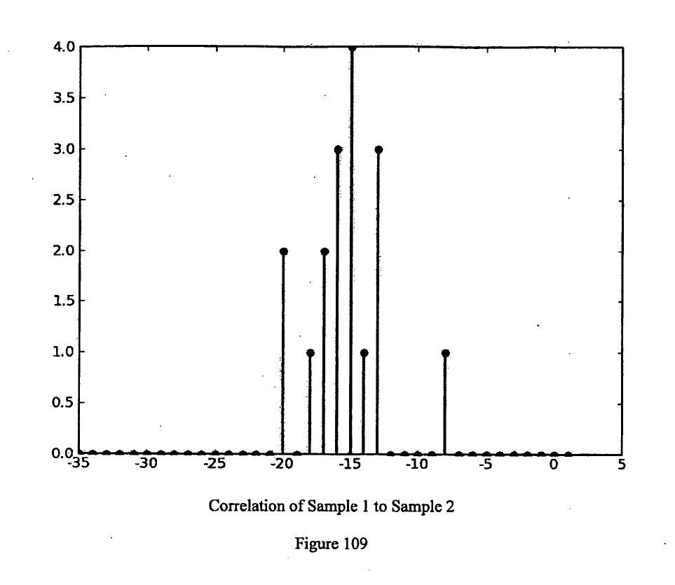

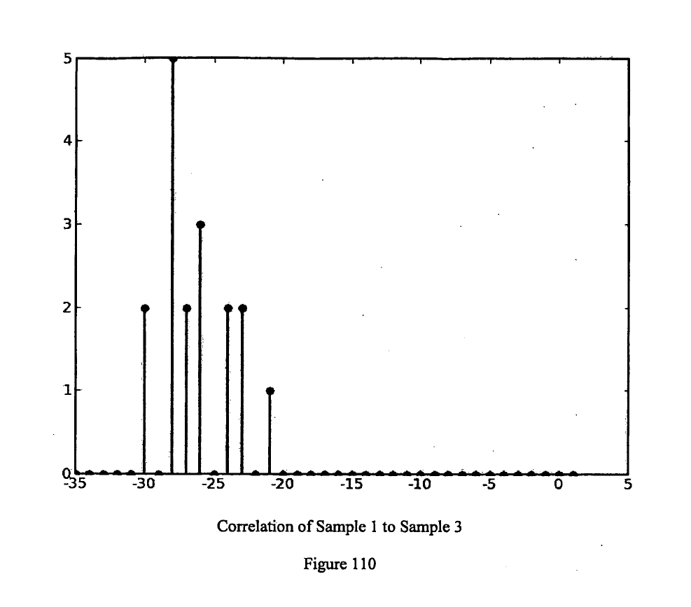

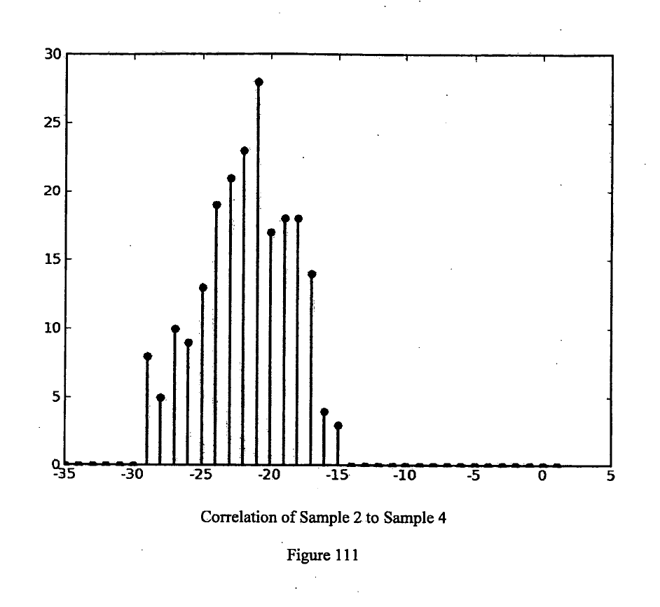

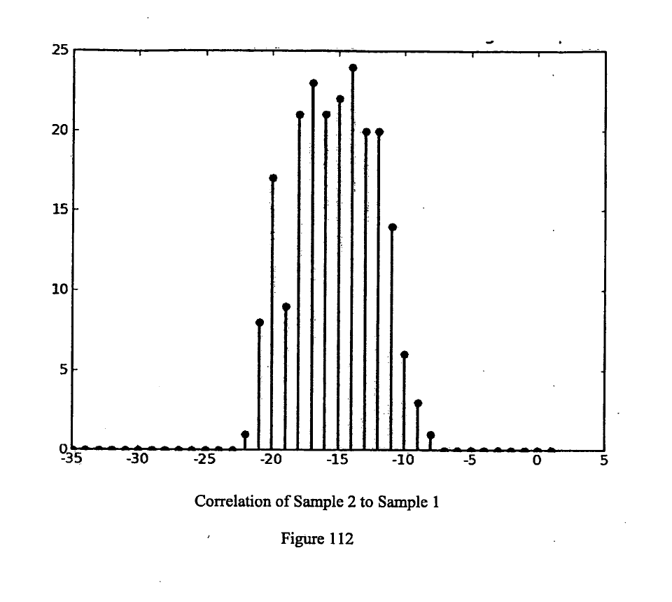

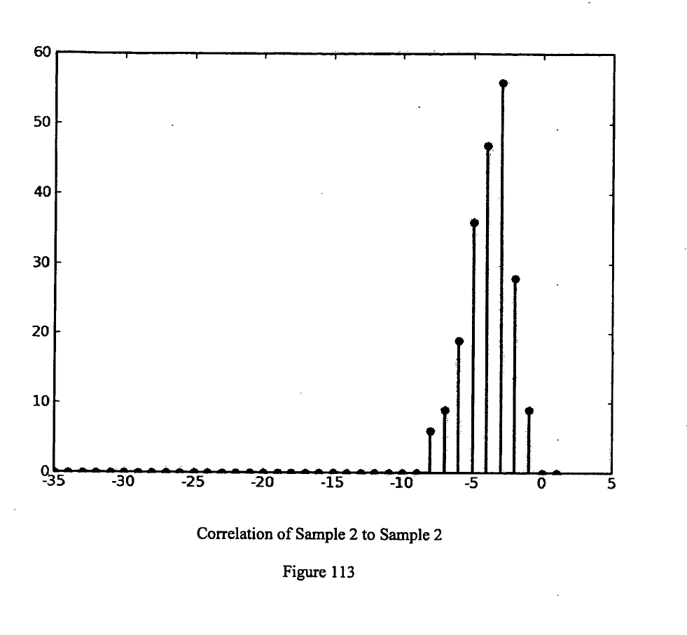

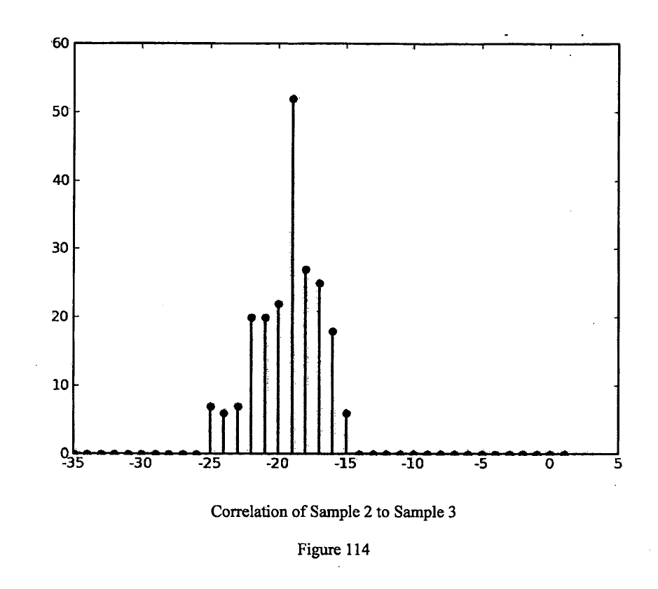

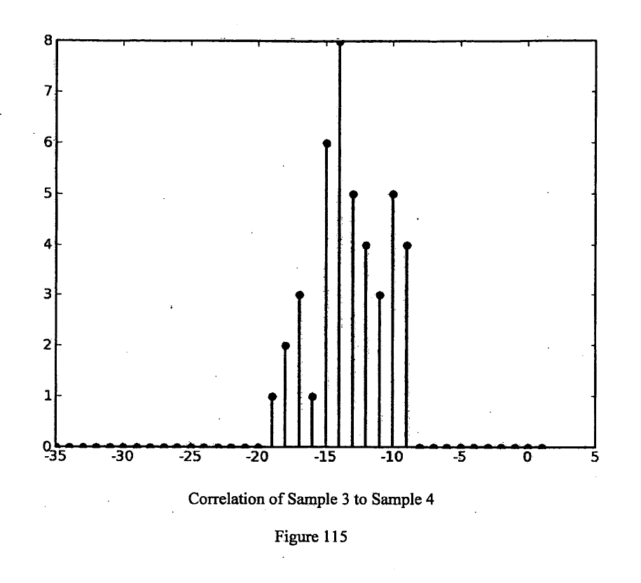

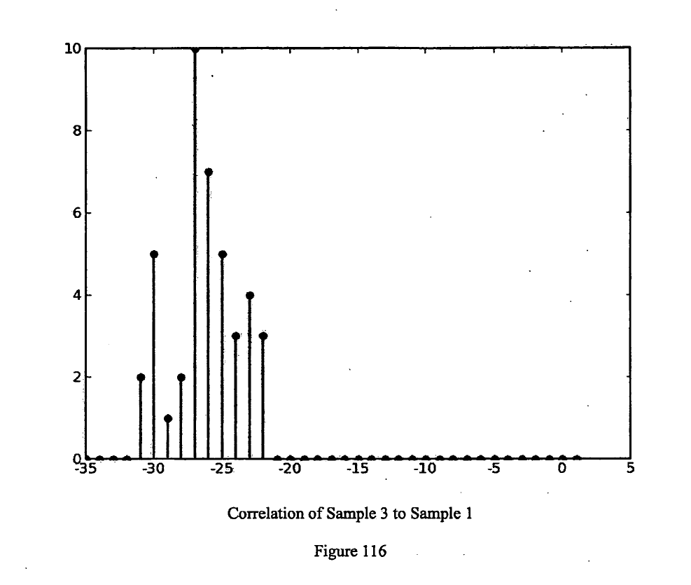

The histograms shown in FIGS. 39-54 show the results achieved using the methods described above.

The methods and results described thus far are all based on using the first derivative of the data curve. Statistical correlation results using the same methods as that used for Test of derivative correlation between full data sets was performed except instead of using the curves of the first derivative of the flow rate versus volume, the flow rate versus volume data curves themselves are used. The histograms shown in FIGS. 55-70 show the results achieved using the methods described above.

The parameterized equation previously presented enabled a non-linear least squares minimization method to determine the parameters of the equation for each curve such that the flow rate versus volume curves were well represented by the parameterized equation. These parameters pᵢ form a coefficient vector that identifies a particular curve. To determine the utility of these coefficient vectors in classifying flow rate versus volume curves, span was augmented to enable statistical correlation coefficients to be calculated using a curves coefficient vectors instead of its data or data derivatives. As with data and data derivative based classification, peak and volume de-rating factors are applied to aid in differentiation. The histograms shown in FIGS. 71-86 show the results achieved using this method.

When fitting a curve to experimental data, one or more of the following methods may be utilized alone or in combination:
self.fitQuality[i].sumSq—sum of squares of difference between average curve and curve i. If the magnitude of this value is greater than some user specified reference, the curve will be marked as failing classification.
self.fitQuality[i].coeffSumsq—sum of squares of difference between coefficients of average curve and curve i. If the magnitude of this value is greater than some user specified reference, the curve will be marked as failing classification.
self.fitQuality[i].distanceFromAvgPeak—distance (along x-axis) between average peak and peak of curve i. If the magnitude of this value is greater than some user specified reference, the curve will be marked as failing classification.
self.fitQuality[i].distanceFromAvgTotalVolume—difference between total volume of the average curve and curve i. If the magnitude of this value is greater than some user specified reference, the curve will be marked as failing classification.
self.fitQuality[i].absDiffFEV1—difference between average FEV1 and FEV1 of curve i. If the magnitude of this value is greater than some user specified reference, the curve will be marked as failing classification.
self.fitQuality[i].absDiffFEV1toFVCratio—difference between FEV1toFVC ratio of the average curve and FEV1toFVCratio of curve i. If the magnitude of this value is greater than some user specified reference, the curve will be marked as failing classification.
self.fitQuality[i].is Bounded—True if curve i is bounded by upper and lower bound curves. False otherwise. Upper and lower bound curves are determined by translating the average curve along the y-axis by the amount self.classificationScaleFactor*peakAvg.y where self.classificationScaleFactor is a user defined parameter and peakAvg.y is the average of the curves peak values.

In this case, a sum of squares was used which is a measure of the quality of the fit of the curve to the data by taking the square root of the sum of the squares of the differences between every point on the curve to every point of the data. This method, or the other listed above, may be used alone or in combination to classify spirometry curves. To implement the method for use in analyzing spirometry data, each curve is compared to the average self (or other) curve and a measure of the likeness of the curves is provided by calculating the square root of the sum of the squares of the differences between them. This difference is subtracted from 1 so all measures for all families of curves have a common upper bound. Peak and volume de-rating are also applied by calculating the square root of the sum of the squares of the differences between the peak and volume of each curve and the average (self or other) peak and average (self or other) volume. Similar to other methods, this method is used for both pruning individual data sets (self-self) and comparing different user's data (self-other). The histograms shown in FIGS. 87-102 display the results achieved using the methods described above.

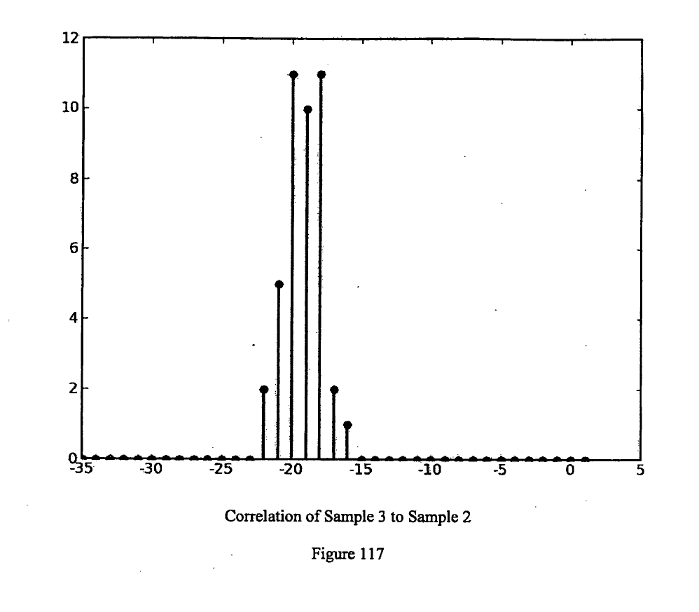

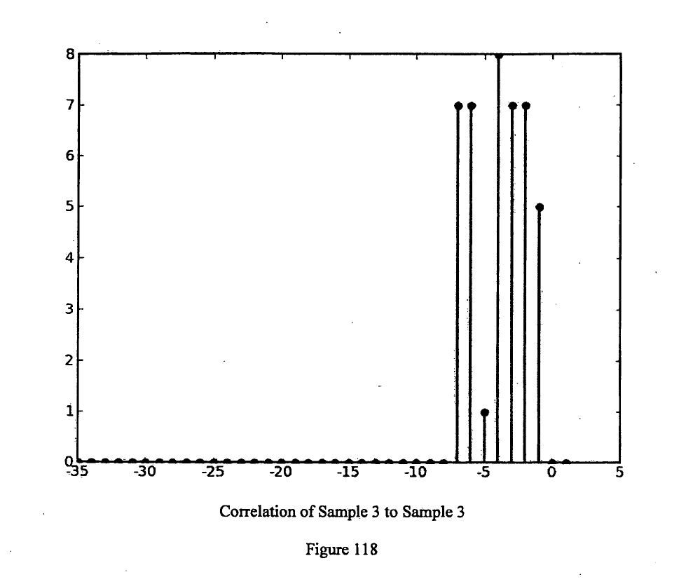

It is evident that the Flow Rate vs. Volume data curves are such that the variation of the Volume value where the Flow Rate peak occurs on each curve is small for a particular user's family of curves. This indicates that a measure of this variation might be useful in classifying the curves. To that end, the square root of the sum of the squares of the differences between the Volume value where the Flow Rate is maximum for each curve is determined and the average of all of these Volume values calculated. Then the sum of the squares of the differences between the Volume where the peak Flow Rate occurs and the average of these Volumes is calculated and contributed to the overall square root of sum of squares of differences calculation as described in the previously. This extra term improves the classification method as can be seen from the histograms shown in FIGS. 103-118. A single factor representing the absolute distance between the points in the graphs in the x, y plane could alternatively be used.

The results described in the examples show that the methods described herein are useful for the classification of spirometry data. The degree to which non-overlapping self-self and self-other distributions overlap can be adjusted by adjusting the pruning parameter, making the pruning algorithm more or less aggressive.

Although the invention has been described with reference to the above example, it will be understood that modifications and variations are encompassed within the spirit and scope of the invention. Accordingly, the invention is limited only by the following claims.

What is claimed is:

1. A method for performing a pulmonary function test comprising verifying the identity of a test patient by comparing pulmonary function test data output for the test patient with reference data of an identified patient using a statistical analysis, thereby verifying the identity of the test patient as the identified patient before the data is further processed or transmitted.

2. The method of claim 1, wherein the statistical analysis comprises:
(a) identifying a peak flow value of an airflow curve generated from data output for the test patient; and
(b) comparing the peak flow value to a peak flow value of an airflow curve generated from reference data for the identified patient.

3. The method of claim 1, wherein the statistical analysis comprises:
(a) normalizing an airflow curve amplitude generated from the data of the test patient to a standard value;
(b) comparing flow-rate values on a point-by-point basis with a normalized reference curve based on reference data of the identified patient to generate point-by-point difference values;
(c) squaring and then summing the point-by-point difference values; and
(d) taking the square root of the sum of the squared point-by-point difference values.

4. The method of claim 1, wherein the statistical analysis comprises:
(a) normalizing an airflow curve amplitude generated from the data of the test patient to a standard value;
(b) shifting the airflow curve to overlay peak flow measurement of the airflow curve with peak flow measurement of reference data for the identified patient;
(c) comparing flow-rate values on a point-by-point basis with a normalized reference curve based on reference data of the identified patient to generate point-by-point difference values;
(d) squaring and then summing the point-by-point difference values; and
(e) taking the square root of the sum of the squared point-by-point difference values.

5. The method of claim 1, wherein the statistical analysis comprises:
(a) decomposing an airflow curve generated from the data output of the test patient into frequency components;
(b) comparing the frequency components from step (a) with frequency components generated from reference data from the identified patient to generate point-by-point difference values;
(c) squaring and then summing the point-by-point difference values; and
(d) taking the square root of the sum of the squared point-by-point difference values.

6. A system for monitoring and collecting pulmonary function test data of a test patient comprising:
(a) an airflow detection device;
(b) a data communications server; and
(c) a computer readable media comprising:
(i) a data structure comprising reference data for an identified patient; and
(ii) commands for performing a statistical algorithm comparing pulmonary function test data of the test patient to the reference data for a patient, wherein the statistical algorithm identifies the test patient as the patient.

7. The system of claim 6, wherein the statistical algorithm comprises:
(a) identifying a peak flow value of an airflow curve generated from the data output for the test patient; and
(b) comparing the peak flow value to a peak flow value of an airflow curve generated from reference data for the identified patient.

8. The system of claim 6, wherein the statistical algorithm comprises:
(a) normalizing an airflow curve amplitude generated from the data of the test patient to a standard value;
(b) comparing flow-rate values on a point-by-point basis with a normalized reference curve based on reference data of the identified patient to generate point-by-point difference values;
(c) squaring and then summing the point-by-point difference values; and
(d) taking the square root of the sum of the squared point-by-point difference values.

9. The system of claim 6, wherein the statistical algorithm comprises:
(a) normalizing an airflow curve amplitude generated from the data of the test patient to a standard value;
(b) shifting the airflow curve to overlay peak flow measurement of the airflow curve with peak flow measurement of reference data for the identified patient;
(c) comparing flow-rate values on a point-by-point basis with a normalized reference curve based on reference data of the identified patient to generate point-by-point difference values;
(d) squaring and then summing the point-by-point difference values; and
(e) taking the square root of the sum of the squared point-by-point difference values.

10. The system of claim 6, wherein the statistical algorithm comprises:
(a) decomposing an airflow curve generated from the data output of the test patient into frequency components;
(b) comparing the frequency components from step (a) with frequency components generated from reference data from the identified patient to generate point-by-point difference values;
(c) squaring and then summing the point-by-point difference values; and
(d) taking the square root of the sum of the squared point-by-point difference values.

11. The system of claim 6, further comprising a computer platform.

12. An airflow detection device comprising:
(a) a data structure comprising reference data for an identified patient; and
(b) commands for performing a statistical algorithm comparing pulmonary function test data of the test patient to the reference data for a patient, wherein the statistical algorithm identifies the test patient as the patient.

13. The device of claim 12, wherein the statistical algorithm comprises:
(a) identifying a peak flow value of an airflow curve generated from the data output for the test patient; and
(b) comparing the peak flow value to a peak flow value of an airflow curve generated from reference data for the identified patient.

14. The device of claim 12, wherein the statistical algorithm comprises:
(a) normalizing an airflow curve amplitude generated from the data of the test patient to a standard value;
(b) comparing flow-rate values on a point-by-point basis with a normalized reference curve based on reference data of the identified patient to generate point-by-point difference values;
(c) squaring and then summing the point-by-point difference values; and
(d) taking the square root of the sum of the squared point-by-point difference values.

15. The device of claim 12, wherein the statistical algorithm comprises:
(a) normalizing an airflow curve amplitude generated from the data of the test patient to a standard value;
(b) shifting the airflow curve to overlay peak flow measurement of the airflow curve with peak flow measurement of reference data for the identified patient;
(c) comparing flow-rate values on a point-by-point basis with a normalized reference curve based on reference data of the identified patient to generate point-by-point difference values;
(d) squaring and then summing the point-by-point difference values; and
(e) taking the square root of the sum of the squared point-by-point difference values.

16. The device of claim 12, wherein the statistical algorithm comprises:
(a) decomposing an airflow curve generated from the data output of the test patient into frequency components;
(b) comparing the frequency components from step (a) with frequency components generated from reference data from the identified patient to generate point-by-point difference values;
(c) squaring and then summing the point-by-point difference values; and
(d) taking the square root of the sum of the squared point-by-point difference values.
